(19) United States
(12) Patent Application Publication
(10) Pub. No.: US 2016/0287741 A1
(43) Pub. Date: Oct. 6, 2016

(54) METASTABLE SILVER NANOPARTICLE COMPOSITES WITH COLOR INDICATING PROPERTIES

(71) Applicants: SIENNA LABS, INC., San Diego, CA (US); NANOCOMPOSIX INC., San Diego, CA (US)

(72) Inventors: Todd J. HARRIS, San Diego, CA (US); Steven J. OLDENBURG, San Diego, CA (US); Richard K. BALDWIN, San Diego, CA (US)

(21) Appl. No.: 15/035,474

(22) PCT Filed: Nov. 18, 2014
(86) PCT No.: PCT/US14/66097

§ 371 Date: May 9, 2016

Related U.S. Application Data
(60) Provisional application No. 61/905,754, filed on Nov. 18, 2013.
(60) Provisional application No. 61/995,494, filed on Apr. 10, 2014.
(60) Provisional application No. 61/998,305, filed on Jun. 24, 2014.

(51) Int. Cl. A61L 15/56; A61L 15/44; A61K 33/38

(52) U.S. Cl. CPC A61L 15/56; A61K 33/38; A61L 15/44

(57) ABSTRACT

Embodiments of the present invention relate to a metastable silver nanoparticle composite, a process for its manufacture, and its use as a source for silver ions and/or colorimetric signaling In various embodiments, the composite comprises, consists essentially of or consists of metastable silver nanoparticles that change shape when exposed to moisture, a stability modulant that controls the rate of the shape change, and a substrate to support the silver nanoparticles and the modulant.

### RELATED APPLICATIONS

This application claims priority to U.S. Ser. No. 61/905,754, filed Nov. 18, 2013; U.S. Ser. No. 61/995,494, filed Apr. 10, 2014; and U.S. Ser. No. 61/998,305, filed Jun. 24, 2014, the contents of each of which are incorporated by reference herein in their entirety.

### BACKGROUND

1. Field of the Invention

The present invention relates to the fields of metal fabrication, color indication, use indication, anti-fouling, ulcer prevention, and infection prevention, in particular compositions and articles containing silver, and processes for the manufacture and use thereof Various embodiments of the present invention relate to a metastable silver nanoparticle composite, a process for its manufacture, and its use as a color indicator. In various embodiments, the composite comprises, consists essentially of, or consists of metastable silver nanoparticles that change shape when exposed to moisture or an etchant, a stability modulant that controls the rate of the shape change, and a substrate to support the silver nanoparticles and the modulant. In various embodiments the composite provides a detectable, e.g., visible, indication of silver nanoparticle shape change, which is useful to signal the status and/or need to replace the composite, or a device or an article in which the silver nanoparticles are incorporated.

2. Description of the Related Art

Silver is a well-known broad-spectrum antimicrobial material. Both ionic and particle (e.g., nanoparticle) forms of silver have been integrated into a number of materials and biomedical devices to increase the efficacy of treatment. For example, Nucryst Pharmaceuticals has developed Acticoat (e.g. U.S. Pat. No. 6,989,156, which is incorporated by reference, in its entirety, herein), which contains nanocrystalline silver that has enhanced solubility and sustained release of silver ions. Other silver dressings have been marketed, including Silvercel™, Aquacel® and Meipex®Ag.

Monitoring changes in local environment (e.g., moisture content, salt, pH, temperature and other factors) is useful in many applications (e.g. indicating continuous use of IV fluids, detecting body moisture, signaling food expiration, and others). Articles incorporating silver have ion release profiles that are a function of their local environment. However, most composites containing colloidal or nanocrystalline silver have color absorbance properties that do not change substantially as silver ions release. Generally, articles and devices incorporating silver presently do not exhibit color changing properties with a high degree of control over color hue, color fidelity, rate of color change, and activation in response to specific local environmental triggers.

### SUMMARY

It is desirable to have the ability to tune or control the ion release profile in order to enhance treatment efficacy and inform when a given article should be replaced. Thus, there is a need in the art for compositions and articles containing silver in a manner such that the release of silver ions is modulated at least in part by the physical and chemical properties of the composite. In one embodiment, the control over the ion release profile is an important factor in the efficacy of treatment. There is a need for a more general class of composites where the time release of silver ions is modulated by the physical and chemical properties of the composite. Provided herein are several embodiments of a composite comprising metastable silver nanoparticles and a stability modulant having antimicrobial activity for use in the prevention of bacterial, fungal and yeast growth. Further, provided herein are several embodiments of a composite comprising metastable silver nanoparticles and a stability modulant, the composite having colorimetric signaling properties useful to indicate the status and/or need to replace the article or device containing the composite.

Color indication, e.g., a detectable color change, is an important characteristic to improve usability and enhance function of various devices or articles. Despite silver's widespread use in medical and consumer products, such products do not generally provide a visible signal of degradation, etching, and ion release. Furthermore, devices and articles have not been described previously that comprise silver particles with absorbance spectral peaks that move through the visible spectrum when activated to provide multi-color read-outs of article status.

The inventors set out to develop antimicrobial silver nanoparticles suitable for incorporation in a wide variety of medical devices and liquid, gel, and solid compositions wherein the time release of silver ions from the nanoparticles could be tuned from rapid to slow in an environment. They discovered that, contrary to previous belief, silver nanoparticles of non-spherical shape, having edges, corners, or vertices of high curvature, when contacted by a solvent, degrade quickly and release ions at a faster rate than silver nanoparticles of similar surface area without high curvature. The amount and rate of silver ion release from these nanoparticles exceeds what would be predicted from a standard surface area model. Without modification as described herein, these silver nanoparticles with edges, corners, or vertices of high curvature degrade quickly, affecting the ability to incorporate such nanoparticles in many medical devices or other compositions in which sustained release is desirable. The inventors have discovered stability modulants including metal oxides, polymers, and salts that, when combined with silver nanoparticles having edges, corners, or vertices of high curvature, create stabilized silver nanoparticles wherein the rate of ion release is decreased relative to silver nanoparticles without stability modulants in a set environment. Thus the present invention provides stabilized silver nanoparticles with high capacity for ion release that offer varying time-release profiles for ion release that are tunable for various applications to achieve an antimicrobial effect. Additionally, as provided herein, the inventors demonstrated that forms of silver nanoparticles, with high curvature and stability modulants, undergo a time and environment dependent shape change to provide a visual indicator of the conditions the nanoparticles, or the article or device in which the nanoparticles are combined with, were exposed to a solvent such as water or a buffered salt solution, while such nanoparticles are substantially stable in the absence of the solvent.

Stabilized silver nanoparticles with edges, corners, or vertices of high curvature have several additional advantages over other materials known in the art including: the efficient production by batch synthesis; the ability to be evenly dispersed in a solution or medium; the ability to be adsorbed or bound onto a surface; the triggered or activated ion release when contacted with solvents or diluents; the colorimetric detection of shifts in shape from high curvature to lower curvature; and the easy incorporation onto or into surfaces of a variety of medical devices, personal care products, household goods and the like, including compositions formulated as liquids, gels, solids, semi-solids, and optionally containing various carriers as provided herein.

Provided herein is a medical device, wherein silver nanoplates are encapsulated by a metal oxide or polymer and localized on or disposed in the surface of the device at a density sufficient to provide an anti-microbial activity or anti-inflammatory activity when activated by a solvent. In some embodiments, the silver nanoplates are localized on or disposed in at least a portion of the device at a concentration sufficient that the color of the silver nanoplates can be readily observed and, optionally, measured. Further, also provided are articles such as medical devices in which a detectable color change occurs upon the silver nanoparticles being contacted with a solvent. In some embodiments the medical device is a tube, syringe, bandage, sheet, sock, sleeve, wrap, shirt, pant, mesh, cloth, sponge, paper adhesive, catheter, orthopedic pin, plate, implant, tracheal tube, insulin pump, wound closure, drain, shunt, dressing, connector, prosthetic device, pacemaker lead, needle, dental prostheses, ventilator tube, ventilator filter, pluerodesis device, surgical instrument, wound dressing, incontinence pad, sterile packaging, clothing, footwear, diaper, sanitary pad, biomedical/biotechnical laboratory equipment, table, enclosure, or wall covering. In some embodiments the medical device is an intravenous (IV) administration set or component thereof, IV extension set, IV connector, IV bag, and the like. In other embodiments the medical device can be modified by the addition of a material or article, typically sterile or sterilized, containing the silver nanoparticles.

Provided herein is an article comprising a material suitable for incorporation into a medical device or article of manufacture, wherein stabilized encapsulated silver nanoplates are disposed on and/or in a surface of the article at a concentration sufficient to provide an anti-microbial activity when activated by a solvent. In some embodiments the article of manufacture is intended for use in a food preparation or storage product, clothing or apparel product, electronic product, a water filtration product, or other durable good.

Provided herein is an antimicrobial composition, comprising a carrier that is a liquid, gel, powder, solid, semi-solid, or emulsion suitable for topical administration and metal oxide or polymer encapsulated silver nanoparticles or nanoplates having at least one vertex, corner, or edge with high curvature.

Provided herein is an antimicrobial composition, comprising a liquid, gel, powder, solid, semi-solid, or emulsion carrier suitable for topical administration and polymer and/or salt stabilized silver nanoplates having at least one vertex, corner, or edge with high curvature. In some embodiments the carrier has a viscosity exceeding 1000 cP enabling the silver nanoplates to be substantially uniformly distributed within the carrier. In some embodiments benefit agents that prolong adherence of silver nanoplates on the skin are added to the composition. In some embodiments the antimicrobial composition is formulated for oral administration, ocular administration, or topical administration. In some embodiments the antimicrobial composition is formulated as a deodorant, antiperspirant, soap, shampoo, moisturizer, or cosmetic, toothpaste, mouthwash or oral hygiene solution, oral tablet, oral extended-release tablet, oral liquid suspension, isotonic and/or lubricant solution for ocular application, lubricant, cream or lotion, surface cleaning agent, laundry detergent, adhesive, or paint.

Provided herein is a formulation comprising stabilized silver nanoplates at one concentration wherein the stabilized silver nanoplates are formulated such that when the formulation is diluted 10 fold the silver nanoplates are susceptible to degradation. Provided herein is a formulation comprising stabilized silver nanoplates wherein the stabilized silver nanoplates are formulated such that when the formulation is exposed to an etchant including a solvent (e.g. water) with salt the silver nanoplates are susceptible to degradation. In some embodiments an applicator is provided wherein stabilized silver nanoparticles are present in a first container and the diluent or etchant is present in a second container, wherein the first container and the second container are operably linked such that the contents thereof are separated by a disruptable separation means.

Provided herein is a composition (also referred to as a composite) comprising metastable silver nanoparticles and a stability modulant having antimicrobial activity for use in the prevention of bacterial, fungal and yeast growth. In some embodiments, the silver nanoparticles of the invention, specifically, nanoparticles having a high curvature, are composites, wherein the nanoparticles have a non-silver core (eg., gold nanorods). As silver ions diffuse away from a silver coated composite, the plasmonic resonance of the silver coating changes and eventually may shift to the plasmonic resonance of the non-silver (e.g., gold) core.

Provided herein in one embodiment is a composite comprising a metastable silver nanoparticle, a stability modulant and a substrate, and where the silver nanoparticles undergo a change in shape when the composite is exposed to moisture or etchant including an etchant that comprises a solvent with salt. In one embodiment the composite provides visible indication of silver nanoparticle shape change to signal the status and/or need to replace the composite or an article in which it is incorporated.

In one embodiment, the silver nanoparticles in the composite are coated with a stability modulant that modifies the silver nanoparticle's ion release rate in a dry environment or a moist environment or in the presence of an etchant including a solvent containing salt.

In one embodiment, the composite contains a coating that is released when the composite is exposed to moisture or etchant, where the released coating modifies the silver nanoparticle's ion release rate in a moist environment or in the presence of an etchant.

In one embodiment the composite contains a coating (e.g. silica oxide, silica) that guides the etching of silver in the composite in a predictable shape when exposed to moisture. In some embodiments the pattern of etching provides a colorimetric signal of the status or expiration (i.e., end of useful life) of the device releasing silver ion.

In one embodiment, the composite contains a stability modulant particle that is bound to the substrate and can dissolve in a moist environment over time to modify the silver nanoparticle's ion release rate in a moist environment. In some embodiments, stability modulants can either be etchants, which include but are not limited to oxidants or protectants which include but are not limited to barriers to prevent silver ion release, reductants, or both. In one embodiment, etchants increase the rate or amount of silver ion release while protectants slow or decrease the amount of silver ion release.

In one embodiment, the color of the composite indicates the concentration and the shape of the silver nanoparticles bound to the substrate.

In one embodiment, the composite is used to treat wounds. In various embodiments, the composite is used to treat wounds, inflammatory skin conditions, mucosal membranes, diseases or conditions of the oral cavity, respiratory disorders, gastrointestinal disorders, nasal disorders, and/or disorders of the urogenital and reproductive systems.

In one embodiment, a composite comprises a metastable silver nanoparticle and a stability modulant, where the silver nanoparticle undergoes a change in shape when the composite is exposed to moisture. In various embodiments, the composite further comprises a substrate. In various embodiments, the silver nanoparticles are nanoplates, nanopyramids, nanocubes, nanorods, or nanowires. In one embodiment, the silver nanoparticles are not spheres and undergo a reduction in aspect ratio when exposed to moisture. In one embodiment, the silver nanoparticles undergo a reduction in aspect ratio when exposed to water or etchant.

In one embodiment, the nanoparticles are faceted and the vertices between their crystal faces undergo an increase in radius of curvature on exposure to moisture. In one embodiment, the stability modulant is a surface coating on the silver nanoparticles. In various embodiments, the surface coating is an oxide, a polymer, organic ligand, thiol, stimulus responsive polymer, polyvinylpyrollidone, silica, polystyrene, tannic acid, polyvinylalcohol, polystyrene or polyacetylene. In one embodiment, the stability modulant is a chemical that is dried onto the substrate. In one embodiment, the chemical is an oxidant. In various embodiments, the chemical is a borate salt, a bicarbonate salt, a carboxylic acid salt, sodium borate, sodium bicarbonate, sodium ascorbate, chlorine salts, primary amines or secondary amines. In one embodiment, the stability modulant is a mixture of etchants and protectants. In one embodiment, the stability modulant is a population of particles. In one embodiment, the particles release chlorine salts or chemicals with primary or secondary amines over a period of time greater than 30 minutes (e.g., 45 minutes, 50 minutes, 60 minutes, 2 hours or more).

In one embodiment, the composite further comprises a protectant on the surface of the particle and a reductant bound to the substrate. In one embodiment, the substrate is a porous network of fibers. In various embodiments, the substrate is a sheet, sock, sleeve, wrap, shirt, pant, mesh, cloth, sponge, paper, filter, medical implant, medical dressing or bandage. In one embodiment, the silver nanoparticles are primarily crystalline. In one embodiment, at least 50% of the silver nanoparticle surface area is a silver ion lattice in the {111} crystal orientation. In one embodiment, the composite releases silver ions over a period of time greater than 30 minutes. In one embodiment, the silver nanoparticles are physisorbed, covalently bonded, or electrostatically bound to the substrate.

In various embodiments, medical device includes a surface for application to a human subject, wherein the surface comprises a plurality of stabilized encapsulated silver nanoplates present at a surface density effective to provide an anti-microbial activity when activated by a solvent. In various embodiments, the surface can comprise any one or more of a metal surface, a plastic surface, a fiber surface, a glass surface, a synthetic bioabsorbable polymer, a naturally derived bioabsorbable polymer. In one embodiment, the surface is inert. In one embodiment, the silver nanoplates are substantially localized on the surface. In one embodiment, the silver nanoplates are substantially disposed in the surface.

In one embodiment, the silver nanoplates are stabilized by encapsulation in a polymer. In various embodiments, the polymer comprises one or more of a polyvinyl polymer, polyvinyl pyrrolidone, polyvinyl alcohol, comprises polyvinyl acrylamide, polystyrene, and/or polyacetylene. In one embodiment, the silver nanoplates are stabilized by encapsulation in a metal oxide. In one embodiment, the silver nanoplates are stabilized by encapsulation in silica. In one embodiment, the silver nanoplates are stabilized by encapsulation in titanium dioxide.

In various embodiments, the solvent comprises water. In one embodiment, the solvent comprises ethanol. In one embodiment, the solvent comprises a body fluid produced by a human subject to which the medical device is applied.

In one embodiment, the silver nanoplates are retained on the surface by adsorption. In one embodiment, the silver nanoplates are retained on the surface by adhesion. In one embodiment, the silver nanoplates are disposed in the surface when the surface is produced. In one embodiment, the silver nanoplates are present on the surface at a surface density of about 0.001 mg to about 1 mg per square inch of surface. In one embodiment, the silver nanoplates are disposed in the surface at a surface density of about 0.001 mg to about 1 mg per square inch of surface.

In various embodiments, the medical device comprises any one or more of a tube, syringe, bandage, sheet, sock, sleeve, wrap, shirt, pant, mesh, cloth, sponge, paper adhesive, catheter, orthopedic pin, plate, implant, tracheal tube, insulin pump, wound closure, drain, shunt, dressing, connector, prosthetic device, pacemaker lead, needle, dental prostheses, ventilator tube, ventilator filter, pluerodesis device, surgical instrument, wound dressing, incontinence pad, sterile packaging, clothing, footwear, diaper, sanitary pad, biomedical/biotechnical laboratory equipment, table, enclosure, or wall covering. In some embodiments the medical device is an IV administration set or component thereof, IV extension set, IV connector, IV bag, and the like.

In one embodiment, silver ions are released into the solvent. In one embodiment, multi-atom silver particles are released into the solvent. In one embodiment, the silver nanoplates have at least one vertex, corner, or edge with high curvature. In one embodiment, the at least one vertex, corner or edge has a radius of curvature that is at least four times smaller than the longest dimension of the silver nanoplate. In one embodiment, the surface is substantially anhydrous prior to use of the medical device.

In various embodiments, the medical device further comprises any one or more of an anti-fungal agent, an anti-microbial agent, an anti-viral agent, or a combination thereof. In various embodiments, the anti-fungal agent is selected from the group consisting of Polyene antifungals, Imidazoles, Triazoles, Thiazoles, Allylamines, Echinocandins, Benzoic acid, Ciclopirox, Flucytosine or 5-fluorocytosine, Griseofulvin, Haloprogin, Polygodial, Tolnaftate, Undecylenic acid, Crystal viol, Piroctone olamine, and Zinc pyrithione; and alternative agents and essential oils

In various embodiments, the anti-microbial agent is selected from the group consisting of alcohols, chorohexadine gluconate, aldehydes, anilides, diamidines, halogen-releasing agents, peroxygen, and/or phenols, bis-biguanide salts, rifampin, minocycline, silver compounds, triclosan, octenidin salts, octenidine dihydrochloride, quaternary ammonium compounds, iron-sequestering glycoproteins, cationic polypeptides, surfactants, zinc pyrithione, broad-spectrum antibiotics, antiseptic agents, and antibacterial drugs

In various embodiments, the anti-viral agent is selected from the group consisting of Abacavir, Aciclovir, Acyclovir, Adefovir, Amantadine, Amprenavir, Ampligen, Arbidol, Atazanavir, Atripla (fixed dose drug), Balavir, Boceprevirertet, Cidofovir, Combivir (fixed dose drug), Darunavir, Delavirdine, Didanosine, Docosanol, Edoxudine, Efavirenz, Emtricitabine, Enfuvirtide, Entecavir, Entry inhibitors, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Tea tree oil, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine

In one embodiment, the stabilized encapsulated silver nanoplates display a visibly detectable color shift when activated by moisture, solvent, or etchant.

In various embodiments, a medical device comprising a surface for application to a human subject, wherein the surface comprises a plurality of stabilized encapsulated silver nanoplates at a surface density sufficient to provide an anti-inflammatory activity when activated by a solvent. In one embodiment, the medical device further comprising an anti-inflammatory agent. In various embodiments, the anti-inflammatory agent is selected from the group consisting of steroids, non-steroidal anti-inflammatory derivatives, immune selective anti-inflammatory derivatives (ImSAIDs), and natural bio-active compounds including Plumbago.

In various embodiments, an article comprises a material suitable for incorporation into a medical device or article of manufacture, the material comprising a surface wherein a plurality of stabilized encapsulated silver nanoplates are disposed substantially on and/or in the surface at a concentration sufficient to provide an anti-microbial activity when activated by a solvent. In various embodiments, the surface comprises a metal, plastic, fiber or glass surface. In various embodiments, the article of manufacture comprises any one or more of a food preparation or storage product, a clothing or apparel product, an electronic product, a water filtration product. In one embodiment, the surface is substantially anhydrous prior to use of the medical device.

In various embodiments, an antimicrobial composition includes a carrier suitable for topical administration to a mammalian subject and a modified silver material comprising a plurality of encapsulated silver nanoplates having at least one vertex, corner, or edge with high curvature. In one embodiment, at least one vertex, corner or edge of the silver nanoplate has a radius of curvature that is at least four times smaller than the longest dimension of the silver nanoplate. In one embodiment, the carrier comprises a liquid, gel, powder, solid, semi-solid, or emulsion. In one embodiment, the carrier comprises a non-aqueous liquid. In one embodiment, the silver nanoplates are encapsulated by a metal oxide. In one embodiment, the silver nanoplates are encapsulated by a polymer. In one embodiment, the antimicrobial composition, when contacted with a solvent, releases silver ions at an enhanced rate relative to a composition of silver nanoparticles without high curvature having about the same or more exposed surface area. In one embodiment, the antimicrobial composition, when contacted with a solvent, releases silver ions at a reduced rate relative to a composition of non-encapsulated silver nanoplates.

In one embodiment, a unit dose containing the composition is in a container for single use. In one embodiment, the container is a glass or polymer vial. In one embodiment, the container further comprises an applicator.

In various embodiments, an actively antimicrobial composition includes a carrier suitable for topical administration to a mammalian subject and a modified silver material comprising a plurality of encapsulated silver nanoparticles having at least one vertex, corner, or edge with a high curvature. In one embodiment, the at least one vertex, corner or edge has a radius of curvature that is at least four times smaller than the longest dimension of the silver nanoplate. In one embodiment, the silver nanoparticle comprises a nanoplate, nanopyramid, nanocube, nanorod, or nanowire. In one embodiment, an antimicrobial composition comprises a carrier suitable for topical administration to a mammalian subject and a modified silver material comprising a plurality of stabilized silver nanoplates having at least one vertex, corner, or edge with high curvature. In one embodiment, the at least one vertex, corner or edge has a radius of curvature that is at least four times smaller than the longest dimension of the silver nanoplate. In one embodiment, the carrier comprises a liquid, gel, solid, semi-solid, or emulsion. In one embodiment, the silver nanoplates are encapsulated by a metal oxide. In one embodiment, the silver nanoplates are encapsulated by a polymer. In one embodiment, the antimicrobial composition, when contacted with a solvent, is capable of releasing silver ions at an enhanced rate relative to a composition of silver nanoparticles without high curvature having about the same or more exposed surface area of silver.

In one embodiment, the antimicrobial composition, when contacted with a solvent, is capable of releasing silver ions at a reduced rate relative to a composition of non-stabilized silver nanoplates. In one embodiment, the carrier has a viscosity exceeding 1000 centipoise (cP). In one embodiment, the silver nanoplates are substantially uniformly distributed within the carrier.

In various embodiments, the stabilized silver nanoplates comprise a borate salt, a bicarbonate salt, a carboxylic acid salt, sodium borate, sodium bicarbonate, sodium ascorbate, chlorine salts, a primary amine or a secondary amine, or a combination thereof In various embodiments, the stabilized silver nanoplates comprise an oxide, a polymer, an organic ligand, a thiol, a stimulus responsive polymer, a polyvinylpyrollidone, silica, tannic acid, polyvinylalcohol, polystyrene or polyacetylene, or a combination thereof In one embodiment, the stabilized silver nanoplates comprise a combination of a polyvinyl polymer and a salt. In one embodiment, the salt comprises a borate salt or a bicarbonate salt. In one embodiment, the stabilized silver nanoplates comprise an etchant. In one embodiment, the stabilized silver nanoplates comprise a protectant.

In one embodiment, a kit comprises the composition and an applicator. In one embodiment, a kit further comprises a solvent and/or etchant. In one embodiment, the solvent and/or etchant and the composition are capable of being mixed in a container.

In various embodiments, an antimicrobial composition includes a carrier suitable for topical administration to a mammalian subject and a modified silver material comprising a plurality of stabilized silver nanoplates having at least one vertex, corner, or edge with high curvature, wherein the composition is suitable for administration to a mammalian subject. In one embodiment, the antimicrobial composition is formulated for oral administration, ocular administration, or topical administration. In one embodiment, the antimicrobial composition is formulated as a deodorant, antiperspirant, soap, shampoo, moisturizer, or cosmetic. In one embodiment, the antimicrobial composition is formulated as a toothpaste, mouthwash or oral hygiene solution. In one embodiment, the antimicrobial composition is formulated as an oral tablet. In one embodiment, the antimicrobial composition is formulated as an oral extended-release tablet. In one embodiment, the antimicrobial composition is formulated as an oral liquid suspension. In one embodiment, the antimicrobial composition is formulated as an isotonic and/or lubricant solution for ocular application. In one embodiment, the antimicrobial composition is formulated as a lubricant. In one embodiment, the antimicrobial composition is formulated as a cream or lotion. In one embodiment, the antimicrobial composition is formulated for human administration. In one embodiment, the antimicrobial composition is formulated for non-human administration. In one embodiment, the antimicrobial composition is formulated as a surface cleaning agent, laundry detergent, adhesive, or paint. In one embodiment, the antimicrobial composition is further comprised of benefit agents that prolong adherence of silver nanoplates on the skin.

In various embodiments, an anti-microbial formulation or color indicating composite comprises stabilized silver nanoplates at a concentration of at least 1 mg/mL, wherein the stabilized silver nanoplates are formulated such that when the concentration thereof is reduced at least 10 fold the encapsulation is susceptible to degradation. In various embodiments, an anti-microbial formulation or color indicating composite comprises stabilized silver nanoplates formulated such that when exposed to an etchant the encapsulation is susceptible to degradation. In one embodiment, the stabilized silver nanoplates are encapsulated by silica. In one embodiment, a kit comprising in one or more containers the formulation and a diluent. In one embodiment, the diluent comprises water, an etchant, or a combination thereof. In one embodiment, the etchant comprises a salt present at a concentration of at least 0.01 mM, 0.1 mM, 0.2 mM, 0.5 mM, 1.0 mM, 5.0mM, 10mM, 50mM, 100mM, 300mM, 500mM or at least 0.001%, 0.01%, 0.05%, 0.1%, 0.45%, 0.9%, 1%, 3%. In one embodiment, the stabilized silver nanoparticles are present in a first container and the diluent is present in a second container, wherein the first container and the second container are operably linked such that the contents thereof are separated by a disruptable separation means. In one embodiment, the kit further includes an applicator. In one embodiment, the disruptable separation means comprises glass or plastic. In one embodiment, the stabilized particles are stable at about 25 degrees C. for at least about 1 week. In one embodiment, the stabilized particles are more stable at about 25 degrees C. than non-stabilized silver nanoplates.

In one embodiment, a composite includes a metastable silver nanoparticle and a stability modulant where the silver nanoparticle undergoes a change in shape when the composite is exposed to moisture or an etchant including a solvent with salt. In one embodiment, the composite includes a substrate. In one embodiment, the silver nanoparticles are nanoplates, nanopyramids, nanocubes, nanorods, or nanowires. In one embodiment, the silver nanoparticles are not spheres and undergo a reduction in aspect ratio when exposed to moisture. In one embodiment, the silver nanoparticles undergo a reduction in aspect ratio when exposed to water. In one embodiment, the nanoparticles are faceted and the vertices between their crystal faces undergo an increase in radius of curvature on exposure to moisture. In one embodiment, the stability modulant is a surface coating on the silver nanoparticles. In one embodiment, the surface coating is an oxide, a polymer, organic ligand, thiol, stimulus responsive polymer, polyvinylpyrollidone, silica, tannic acid, polyvinylalcohol, polystyrene or polyacetylene. In one embodiment, the stability modulant is a chemical that is dried onto the substrate. In one embodiment, the chemical is an oxidant. In one embodiment, the chemical is a borate salt, a bicarbonate salt, a carboxylic acid salt, sodium borate, sodium bicarbonate, sodium ascorbate, chlorine salts, primary amines or secondary amines. In one embodiment, the stability modulant is a mixture of etchants and protectants. In one embodiment, the stability modulant is a population of particles. In one embodiment, the particles release chlorine salts or chemicals with primary or secondary amines over a period of time greater than 30 minutes. In one embodiment, there is a protectant on the surface of the particle and a reductant bound to the substrate. In one embodiment, the substrate is a porous network of fibers. In one embodiment, the substrate is a sheet, sock, sleeve, wrap, shirt, pant, mesh, cloth, sponge, paper, filter, medical implant, medical dressing or bandage. In one embodiment, the silver nanoparticles are primarily crystalline. In one embodiment, at least 50% of the silver nanoparticle surface area is a silver ion lattice in the {111} crystal orientation. In one embodiment, the composite releases silver ions over a period of time greater than 30 minutes. In one embodiment, the silver nanoparticles are physisorbed, covalently bonded, or electrostatically bound to the substrate.

### BRIEF DESCRIPTION OF THE DRAWINGS

Further objects, features and advantages of the invention will become apparent from the following detailed description taken in conjunction with the accompanying figures showing illustrative embodiments of the invention, in which the following is a description of the drawings. The drawings are examples, and should not be used to limit the embodiments. Moreover, recitation of embodiments having stated features is not intended to exclude other embodiments having additional features or other embodiments incorporating different combinations of the stated features. Further, features in one embodiment (such as in one figure) may be combined with descriptions (and figures) of other embodiments.

FIG. 1A illustrates one embodiment of a cubic nanoplate that has a small radius of curvature.

FIG. 1B illustrates one embodiment of a cubic nanoplate with a larger radius of curvature.

FIG. 2A illustrates one embodiment of a generally plate shaped nanoparticle with a specific width and thickness.

FIG. 2B illustrates a one embodiment of a change of shape into another particle that has an increased thickness and a decreased width.

FIG. 3 illustrates the optical spectra of one embodiment of silver nanoplates that have different aspect ratios.

FIG. 4 shows a transmission electron microscopy (TEM) image of one embodiment of silver nanoplates after synthesis.

FIG. 5 shows a TEM image of one embodiment of silver nanoplates after five days.

FIG. 6 shows a chart that documents the optical shift associated with the shape change of silver nanoplates according to one embodiment of the invention.

FIG. 7A illustrates one embodiment of a composite that contains fibers and metastable silver particles.

FIG. 7B shows metastable silver particles that are plate shaped according to one embodiment of the invention.

FIG. 7C shows metastable silver particles that are plate shaped and coated with a stability modulant according to one embodiment of the invention.

FIG. 8A illustrates a one embodiment of a composite that contains fibers, metastable silver particles and a chemical stabilant.

FIG. 8B illustrates the chemical coating component that is applied to the fiber and nanoparticles to form the composite according to one embodiment of the invention.

FIG. 9 illustrates a composite that contains fibers, metastable silver particles and particles that release a stability modulant over time according to one embodiment of the invention.

FIG. 10A illustrates a bandage that contains metastable silver particles attached to a woven mesh according to one embodiment of the invention.

FIG. 10B illustrates a close-up view of the metastable silver particles attached to a woven mesh according to one embodiment of the invention.

FIG. 11 shows a chart that documents the enhanced ion release properties of a silver nanoplate according to one embodiment of the invention, with a high curvature relative to a silver spherical nanoparticle with normalized surface area.

FIG. 12 shows the ion release from concentrated silver nanoplates according to one embodiment of the invention, stabilized with borate and a polyvinyl polymer (PVP) diluted 200-fold with water or diluted 200-fold with 5 mM borate. In contact with a solvent in the absence of the borate stabilant, the silver nanoplates rapidly release silver ions whereas in the presence of the both PVP and borate stabilant, the silver nanoplates retain their shape and do not appreciably release silver ions even at 200 fold reduced concentration.

FIG. 13 shows colorimetric signaling of silver nanoplates in solution. Silver nanoplates change color from blue to violet to red/orange while actively releasing ions. Yellow color signals complete color change, indicating that all or most of the silver nanoplates have become substantially spherical, or have otherwise achieved a stable shape and therefore susceptible to reduced or no further silver ion release.

FIG. 14 shows free silver ion release profiles for various silver nanoparticles solutions. Silver nanoplates with high curvature have higher ion release potential compared to silver nanospheres with low curvature at the same concentration. 5 mM borate acts to stabilize silver nanoplate ion release providing a negative control.

FIG. 15 shows colorimetric signaling from silver nanoplates embedded in a wound dressing. Silver nanoplates in the bed of an adhesive bandage result in the appearance of a blue color, which changes to red/orange and then to yellow/clear after moisture exposure, signaling activation of ion release and the corresponding shape change of the silver nanoparticles, eventually resulting in a completion of the change in silver nanoparticle shape and corresponding loss of further antimicrobial activity.

FIG. 16 shows a transmission electron micrograph (TEM) of individual stabilized encapsulated (silica coated) silver nanoplates before and after etching. In the presence of 0.9% NaCl silver is etched out of the silica shell leaving a much smaller diameter silver plate adjacent to a void space where the larger diameter silver core was previously. The dimensions of the initial and etched cores give color properties of blue and yellow to their respective solutions. The Silica shell stabilizes and guides etching so that the particles display unique color signatures as they degrade.

FIG. 17 shows colorimetric signaling from silver nanoplates embedded in a foam dressing. Silver nanoplates color the foam blue and change to purple, then red/orange and then to yellow/clear after exposure to wound exudate signaling strikethrough/leakage of exudate and activation of ion release and silver nanoparticle shape change. Strikethrough signaling occurs as a function of the rate of exudate produced by the wound. The vivid color changes that occur on the bandage after strikethrough inform a patient or caregiver of appropriate times to change the dressing.

FIG. 18 shows colorimetric signaling from silver nanoplates embedded in silicone. Silver nanoplates color the silicone blue and change to purple within a day and continue to change to red, orange, yellow and eventually white. These silicones can be placed in IV connectors or other administration set components to signal useful lifetimes and antimicrobial activity of components in the IV line.

FIG. 19 shows a CAD rendering of a continuous use indicator unit to be incorporated into an IV set, needless connector, or catheter. A plastic housing (191 & 193) incases a silicone strip (192) allowing IV fluid running through the unit to pass over the silicone. Embedded into the silicone are silver nanoplates that change color over time with exposure to salts in the IV fluid.

FIG. 20 shows an color changes from a silicone strip containing silver nanoplates after exposure to saline for 0, 24, 48, 72 and 86 hours. The silicone strip changes from blue to purple and eventually to red indicating precisely how long the unit has been in continuous use.

FIG. 21 shows a rendering of a continuous use indicator unit with a legend printed on the outer surface where colors corresponding to each use day are provided and labeled with the corresponding day. The indicator itself is printed onto white silicone in a detectable shape (shield) so that an operator can differentiate the color of the indicator from blood or other potential contaminates in the IV line.

FIG. 22 shows colorimetric signaling from silver nanoplates embedded in discreet spots of an article for body moisture indication from a mammalian subject. Silver nanoplates color spots on the article blue and change to purple, then red/orange and then to yellow/clear after exposure to sweat, exudate, or incontinence. The vivid color changes that occur on the article inform a patient or caregiver that moisture is present and how long the moisture has been there. Useful articles include undergarments, seat or bed covers/linens, diapers, socks, outer garments, or other articles.

### DETAILED DESCRIPTION OF THE PREFERRED EMBODIMENT

Several embodiments of this invention include a composite that when exposed to moisture releases silver ions. In various embodiments, the composite comprises, consists essentially of, or consists of metastable silver nanoparticles, a stability modulant and a substrate. In various embodiments, the composite comprises, consists essentially of, or consists of metastable silver nanoparticles (silver nanoplates or silver nanoparticles with at least one vertex, corner or edge with high curvature), a stability modulant, and a substrate (including a surface or carrier).

As used herein, the terms and phrases set out below have the meanings which follow:

“Anti-microbial effect” means that atoms, ions, molecules, clusters, or multi-atom particles of the anti-microbial metal (hereinafter “species” of the anti-microbial metal) are released into the solvent including water, an alcohol, or water based electrolyte which the material contacts in concentrations sufficient to inhibit bacterial (or other microbial) growth in the vicinity of the material. The most common method of measuring anti-microbial effect is by measuring the zone of inhibition (ZOI) created when the material is placed on a bacterial lawn. A relatively small or no ZOI (ex. less than 1 mm) indicates a non useful anti-microbial effect, while a larger ZOI (ex. greater than 5 mm) indicates a highly useful anti-microbial effect.

“Silver nanoplates” means nanoparticles substantially composed of silver metal formed in a shape characterized by lengths along the three principle axes wherein the axial length of two of the principle axes is at least two times greater than the axial length of the shortest principle axis and the shortest principal axial length is less than about 500 nm. Silver nanoplates have a variety of different cross sectional shapes including circular, triangular, or shapes that have any number of discrete edges. At least one vertex, edge, or corner of silver nanoplates have high curvature or a small radius of curvature relative to the largest dimension of the particle causing them to be metastable nanoparticles with respect to shape. By definition, a silver nanoplate has at least one vertex, corner or edge with radius of curvature that is four times smaller than the longest dimension of the silver nanoplate.

“Radius of curvature” of a vertex, edge or corner of a nanoparticle is defined to be the radius of a circle that best matches the outer dimensions of a cross sectional cut through a vertex, edge, or corner of the nanoparticle.

“Metastable nanoparticles with respect to shape” or “Metastable nanoparticles” refers to nanoparticles of a determined size and shape, the shape and size of which do not vary substantially under one set of environmental conditions, and which undergo a size and/or shape change under another set of environmental conditions. Examples of shape changes include a reduction in aspect ratio, a change in the local radius of curvature at the vertex between two crystal faces, a transformation to a more spherical shape, the deposition of metal ions onto one or more surfaces of the nanoparticle, or a change in the surface roughness of the particle. Shape changes may coincide with the release of silver species in the solvent in which a nanoparticle contacts producing an anti-microbial effect. Silver nanoplates are metastable nanoparticles with respect to shape as are other silver nanoparticles formed in shapes with high curvature including oblate and prolate spheroids, flakes, discs, rods, wires, triangular, pyramidal, bipyrimidal, cubes, and other crystalline shapes. For clarity, the term “metastable nanoparticles,” encompasses and is interchangeable with “silver nanoplates”, “silver materials” and “silver nanoparticles” having “at least one vertex, corner, or edge with high curvature”.

“Sustained release” or “sustainable basis” are used to define release of atoms, molecules, ions or clusters of an anti-microbial metal that continues over time measured in hours or days, and thus distinguishes release of such metal species from the bulk metal, which release such species at a rate and concentration which is too low to achieve an anti-microbial effect, and from highly soluble salts of anti-microbial metals such as silver nitrate, which releases silver ions virtually instantly, but not continuously, in contact with a solvent including water, an alcohol, or water based electrolyte.

“Triggered release,” “triggered”, or “activated” is used to define release of atoms, molecules, ions or clusters of an anti-microbial metal triggered by a change in environmental conditions. Triggered release can cause a release of anti-microbial species virtually instantly or initiate a sustained release of anti-microbial species from a silver nanoparticle.

“Shape instable silver nanoplate” refers to a silver nanoplate which undergoes a detectable size and/or shape change rapidly in a set of environmental conditions, the rapidity of such change able to be modulated as provided herein and as otherwise recognized by one skilled in the art.

“Encapsulate” or “encapsulation” means covering or coating a substantial portion of a material; an “encapsulant” is the product of the encapsulation process, and may refer to the covering or coating, or the coating and the coated material.

An “etchant” means a solvent, or a combination of solvent(s) and solule(s), that promotes or accelerates the dissolution of an ion (e.g., a silver ion) from a particle or solid material (e.g., a silver nanoplate). Also included as etchants are salts, salt-containing materials, and salts dissolved into a solvent. Further included as etchants are highly pure or pure solvent (e.g., water) solutions that extract ions from materials.

“Stability modulant” is an additive to a composition or an environment containing a silver nanoplate such that a silver nanoplate in contact with a solvent, releases atoms, ions, molecules or clusters containing silver into the solvent at a reduced rate relative to the composition or environment without the stability modulant. Stability modulants are coatings that encapsulate the silver nanoplates or a set of additives dispersed in a composition comprising a silver nanpolate. Stability modulants may be used to achieve sustained release or triggered release from silver nanoplates.

“Stabilized silver nanoplate” refers to a silver nanoplate in a composition or environment with a stability modulant that causes the silver nanoplate, in contact with a solvent, to release atoms, ions, molecules or clusters containing silver into the carrier at a reduced rate relative to a composition or environment without the stability modulant.

“Encapsulated silver nanoplate or “stabilized encapsulated nanoplate” refers to a silver nanoplate coated or encapsulated by a stability modulant that causes the silver nanoplate, in contact with a solvent, to release atoms, ions, molecules or clusters containing silver into the carrier at a reduced rate relative to a silver nanoplate that is not encapsulated.

“Biocompatible” means non-toxic for the intended utility. Thus, for human utility, biocompatible means non-toxic to humans to human tissues.

“Medical device” means any device, appliance, fixture, fiber, fabric or material intended for a medical, health care or personal hygiene utility, including, without limitation orthopaedic pins, plates, implants, tracheal tubes, catheters, insulin pumps, wound closures, drains, shunts, dressings, connectors, prosthetic devices, pacemaker leads, needles, dental prostheses, ventilator tubes, surgical instruments, wound dressings, incontinent pads, sterile packaging clothing footwear, personal hygiene products such as diapers and sanitary pads, and biomedical/biotechnical laboratory equipment such as tables, enclosures and wall coverings and the like. Medical devices may be made of any suitable material, for example metals, including steel, aluminum and its alloys, latex, nylon, silicone, polyester, glass, ceramic, paper, cloth and other plastics and rubbers. For indwelling medical devices, the device will be made of a bioinert or biocompatible material. The device may take on any shape dictated by its utility, ranging from flat sheets to disc, rods and hollow tubes. The device may be rigid or flexible, a factor dictated by its intended utility.

“Alcohol or water based electrolyte” is meant to include any alcohol or water based electrolyte that the anti-microbial materials of the present invention might contact in order to become activated, i.e., the release of species of the anti-microbial metal into a solution containing the electrolyte. The term is meant to include alcohols, water, gels, fluids, solvents, and tissues containing water, including body fluids (for example blood, urine or saliva), and body tissue (for example skin, muscle or bone).

“Color change” is meant to include changes of intensity of light under monochromatic light as well as changes of hue from white light containing more than one wavelength. A “color indicator” or a “colorimetric display” includes any article, device, component, compound or physical material that indicates a color change or has a property of a color change.

A “curable liquid” is meant to include any liquid material, typically containing one or more polymers capable of thermosetting to form a solid, the process of thermosetting being termed a “cure” or “curing”. Curable liquids also include polymers capable of being cross-linked to form a solid.

“Partly light transmissive” when used to describe a thin film of the top layer material means that the thin film is capable of transmitting at least a portion of incident visible light through the thin film.

“Detectable” when used to describe a color change means an observable shift in the dominant wavelength of the reflected light, whether the change is detected by instrument, such as a spectrophotometer, or by the human eye, particularly an unaided human eye. The dominant wavelength is the wavelength responsible for the colour being observed.

“Use” or “Use indication” is meant to describe various forms of monitoring or signaling how an article or product is being used. The term may include use time of an article (e.g., the time since activated or put in use). The term may refer to the detection of an activation step or steps, such as moisture or salt exposure. Use indication may refer to the type or amount of exposure (e.g. salt concentration, pH) and/or the amount of time over which the exposure occurred.

“Wound” means cut, lesion, burn or other trauma to human or animal tissue requiring a wound dressing.

“Wound dressing” means a covering for a wound.

“Bioabsorbable materials” are those useful in medical devices or parts of medical devices, that is which are biocompatible, and which are capable of bioabsorption in a period of time ranging from hours to years, depending on the particular application.

“Bioabsorption” means the disappearance of materials from their initial application site in the body (human or mammalian) with or without degradation of the dispersed polymer molecules.

“Biocompatible” means generating no significant undesirable host response for the intended utility.

“Therapeutically effective amount” is used herein to denote any amount of a formulation of the silver nanoplates which will exhibit an antiproliferative effect, anti-inflammatory effect, or anti-microbial effect. A single application of the formulations of the present invention may be sufficient, or the formulations may be applied repeatedly over a period of time, such as several times a day for a period of days or weeks. The amount of the active ingredient, that is the silver nanoplates in the form of a coating, powder or dissolved in a liquid, gelled, or solid carrier, will vary with the conditions being treated, the stage of advancement of the condition, and the type and concentration of the formulation being applied. Appropriate amounts in any given instance will be readily apparent to those skilled in the art or capable of determination by routine experimentation.

“Anti-inflammatory effect” means a reduction in one or more of the symptoms of erythema (redness), edema (swelling), pain and pruritus which are characteristic of inflammatory skin conditions.

“Inflammatory skin conditions” refers to those conditions of the skin in which inflammatory cells (e.g., polymorphonuclear neutrophils and lymphocytes) infiltrate the skin with no overt or known infectious etiology, but excluding psoriasis and its related conditions. Symptoms of inflammatory skin conditions generally include erythema (redness), edema (swelling), pain, pruritus, increased surface temperature and loss of function. As used herein, inflammatory skin conditions include, but are not limited to, eczema and related conditions, insect bites, erythroderma, mycosis fungoides and related conditions, pyoderma gangrenosum, erythema multiforme, rosacea, onychomycosis, and acne and related conditions, but excluding psoriasis and its related conditions.

“Hydrocolloid” means a synthetically prepared or naturally occurring polymer capable of forming a thickened gel in the presence of water and polyols (swelling agent). The swelling agent must be capable of swelling the hydrocolloid chosen in order to form the gel phase.

“Hydrogels” means a hydrocolloid swollen with water or another hydrophilic liquid which is used for absorbing or retaining moisture or water.

“Gel” means a composition that is of suitable viscosity for such purposes, e.g., a composition that is of a viscosity that enables it to be applied and remain on the skin.

“Carrier” means a suitable vehicle including one or more solid, semisolid, gel, or liquid diluents, excipients or encapsulating substances which are suitable for topical administration to a mammalian subject.

“Composite” refers to the composition comprising both a silver nanoparticle and a stability modulant.

“Substrate” refers to a surface of an article or a carrier.

“Mucosal membrane” includes the epithelial membranes which line the oral cavity, the nasal, bronchial, pulmonary, trachea and pharynx airways, the otic and ophthalmic surfaces, the urogenital system, including the prostate, the reproductive system and the gastrointestinal tract, including the colon and rectal surfaces. Reference to mucosal membrane herein is meant to include the surface membranes or cell structures of the mucosal membrane at a targeted site.

“Diseases or conditions of the oral cavity” means diseases or conditions of the oral cavity whether infectious, inflammatory or immunologic in origin, including without limitation periodontal disease, gingivitis, periodontitis, periodontosis, inflammatory conditions of the tissues within the oral cavity, caries, necrotizing ulcerative gingivitis, oral or breath malodor, herpetic lesions, infections following dental procedures such as osseous surgery, tooth extraction, periodontal flap surgery, dental implantation, scaling and root planing, dentoalveolar infections, dental abscesses (e.g., cellulitis of the jaw; osteomyelitis of the jaw), acute necrotizing ulcerative gingivitis, infectious stomatitis (i.e., acute inflammation of the buccal mucosa), Noma (i.e., gangrenous stomatitis or cancrum oris), sore throat, pharyngitis, and thrush.

“Respiratory disorders” means respiratory disorders of the nasal, bronchial, pulmonary, trachea and pharynx airways whether infectious, inflammatory or immunologic in origin, including without limitation emphysema, chronic bronchitis, asthma, pulmonary edema, acute respiratory distress syndrome, bronchopulmonary dysplasia, pulmonary fibrosis, pulmonary atelectasis, tuberculosis, pneumonia, TENS, Stevens Johnstone Syndrome, common cold, sore throat, pharyngitis, and cystic fibrosis.

“Gastrointestinal disorders” means disorders of the gastrointestinal tract whether infectious, inflammatory or immunologic in origin, including without limitation, digestive ulcers such as esophageal ulcer, gastric ulcer and duodenal ulcer, and also esophagitis, gastritis, enteritis, enterogastric intestinal hemorrhage, colitis, inflammatory bowel disease, and hemorrhoids.

“Nasal disorders” means disorders of the nasal passages whether infectious, inflammatory or immunologic in origin, including without limitation sinusitis.

“Disorders of the urogenital and reproductive systems” means disorders of these systems whether infectious, inflammatory or immunologic in origin, including without limitation STD's, HIV, chlamydia, syphilis, gonorrhea, Herpes, genital warts, and prostatitis.

“Moist” means an environment that is high in humidity (>50% RH) or is characterized by the presence of liquid water, and “moisture” includes liquid water and any solution containing liquid water.

Silver Nanoplates and Silver Nanoparticles with High Curvature

Metastable silver nanoparticles can be any shape. In certain embodiments the metastable silver nanoparticles have a non-spherical shape. In various embodiments, shapes that may be metastable include spheres, plates, discs, rods, wires, triangular, pyramidal, bipyrimidal, cubes, and other crystalline faceted shapes. In one embodiment, at least one vertex, edge, or corner of a silver nanoparticle has high curvature or a small radius of curvature relative to the largest dimension of the particle causing them to be metastable nanoparticles with respect to shape. In various embodiments, silver nanoplates of high curvature may include nanoplates, nanopyramids, nanocubes, nanorods, or nanowires.

In one embodiment a substantial portion of the metastable silver nanoparticles have a plate shape and are referred to as nanoplates. In one embodiment, silver nanoplates are characterized by lengths along the three principle axes wherein the axial length of two of the principle axes (e.g., edge length) is at least two times greater than the axial length of the shortest principle axis (e.g., thickness) and the shortest principal axial length is less than about 500 nm (e.g., 400 nm, 300 nm, 250 nm, 100 nm or less) and greater than zero (e.g., 0.5 nm, 1 nm, 5 nm, or more) or any range therein. In some embodiments the shortest principal axial length is from 0.5 nm to 2 nm, 1 nm to 5 nm, 2 nm to 10 nm, 2 nm to 30 nm, 5 nm to 30 nm, 10 nm to 50 nm, 50 nm to 100 nm, 100 nm to 500 nm, or any range therein. In one embodiment, a silver nanoplate has at least one vertex, corner or edge with radius of curvature that is four times smaller than the longest dimension of the silver nanoplate.

In various embodiments, silver nanoplates have a variety of different cross sectional shapes including circular, triangular, or shapes that have any number of discrete edges. In one embodiment the nanoplates have less than 20, 15, 10, 8, 6, 5, or 4 edges (e.g., 3 edges, 2, edges, 1 edges). In one embodiment the nanoplates have more than 2, 3, 4, or 5 edges (e.g., 7, 8, 12, 17 or more edges). In some embodiments the silver nanoplates have relatively sharp corners and in some embodiments the corners are relatively rounded.

In some embodiments of silver nanoplates, there are a variety of different cross sectional shapes within the same sample. In other embodiments of silver nanoplate solutions greater than 5%, 10%, 20%, 30%, 40%, 50%, 60%, 70%, 80%, or 90% of the number of particles in solution are silver nanoplates with the other particles having different shapes including, but not limited to, spherical, cubic, and/or irregular. In some embodiments the nanoplates have one or two flat sides. In one embodiment the nanoplates are pyramidal. In some embodiments the particles are primarily crystalline. In some embodiments at least 10%, 20%, 50%, 75% or 90% (e.g., 15%, 55%, 95%) of the silver nanoparticle surface is in the { 111 } crystal orientation.

In one embodiment, the nanoparticles have a rod shape. Silver rods are characterized by lengths along the three principle axes wherein the axial length of one of the principle axes is at least about two times greater than the axial length of the other two principle axis and the shortest principal axial length is less than about 500 nm (e.g., 400 nm, 300 nm, 250 nm, 100 nm or less) and greater than zero (e.g., 0.5 nm, 1 nm, 5 nm, or more) or any range therein.

In one embodiment, the nanoparticles have a cubic shape. Cubes have six flat generally equal faces. In some embodiments the faces of the cubes meet at a sharp edge. In other embodiments the edges where two faces meet are rounded. In other embodiments the corners of the cubes are rounded. The radius of curvature of the edges or corners is defined to be the radius of a circle that best matches the outer dimensions of a cross sectional cut through the vertex, edge or corner of the cube.

In one embodiment, the nanoparticles have multiple facets or sides. In some embodiments a side has a surface roughness less than 10%. The edges or vertices of the faces can have different radii of curvature. In one embodiment a nanoparticle is pyramidal in shape where the figure has a polygonal base and triangular faces that meet at a common point. In one embodiment the shape of the particles is a bipyramid that consists of two pyramids with a common polygonal base.

In one embodiment, the metastable silver nanoparticles are generally spherical. The silver nanoparticles change shape by decreasing in size over time in the presence of stability modifiers.

In one embodiment, the aspect ratio of a nanoparticle is referred to as the ratio between the longest principal axis (e.g., edge length) and the shortest principal axis (e.g., thickness). In one embodiment the average aspect ratio of the metastable nanoparticles is greater than about 1.5, 2, 3, 4, 5, 7, 10, 20, 30, or 50 (e.g., 15, 25, 60, 100 or more). In one embodiment the average aspect ratio of the metastable nanoparticles is between 1.5 and 25, 2 and 25, 1.5 and 50, 2 and 50, 3 and 25, or 3 and 50 (e.g, 10 and 15, 12 and 17, 35 and 45, etc.). In various embodiments, the nanoparticle has edge lengths less than about 500 nm, 250 nm, 200 nm, 150 nm, 100 nm, 80 nm, 60 nm or 50 nm. In various embodiments, the nanoparticle has edge lengths greater than about 5 nm, 10 nm, 20 nm, 30 nm, 50 nm or 100 nm. In one embodiment the nanoparticle has a thickness (third principle axis) that is less than about 500 nm, 300 nm, 200 nm, 100 nm, 80 nm, 60 nm, 50 nm, 40 nm, 30 nm, 20 nm, or 10 nm. In one embodiment the thickness of the nanoplates is between 1 nm and 20 nm, 2 nm and 50 nm, 5 nm and 20 nm, 5 nm and 50 nm, and 5 nm and 100 nm.

In an embodiment, the silver nanoparticles are metastable with respect to their shape. Metastable nanoparticles have a fixed size and shape under one set of environmental conditions but then undergo a size or shape change under another set of environmental conditions. In various embodiments, examples of shape changes include a reduction in aspect ratio, a change in the local radius of curvature at the vertex between two crystal faces, a transformation to a more spherical shape, the deposition of metal ions onto one or more surfaces of the nanoparticle, or a change in the surface roughness of the particle. In an embodiment, the silver nanoparticles have a high aspect ratio or highly faceted shape and when exposed to moisture silver ions from one portion of the nanoparticle are released into solution and redeposit on another portion of the particle. In one embodiment the silver nanoparticles are plate shaped and the primary dissociation of the silver ions occurs at the edges of the particle and is deposited primarily onto the faces of the nanoparticle which reduces the aspect ratio of the particle. In an embodiment, the silver nanoparticles have a rod or wire shape and in a moist environment, silver ions are released from the ends of the rods or wires and deposit onto the long axis surface of the particles resulting in a reduced aspect ratio.

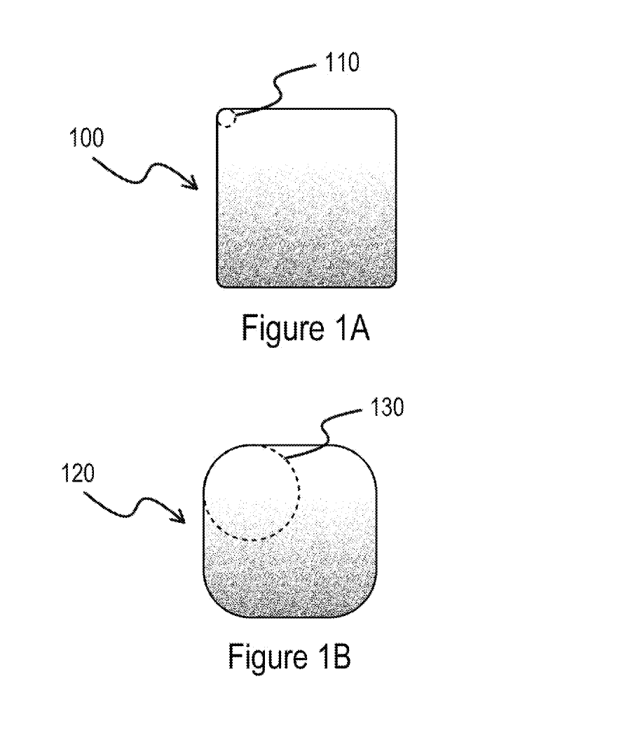

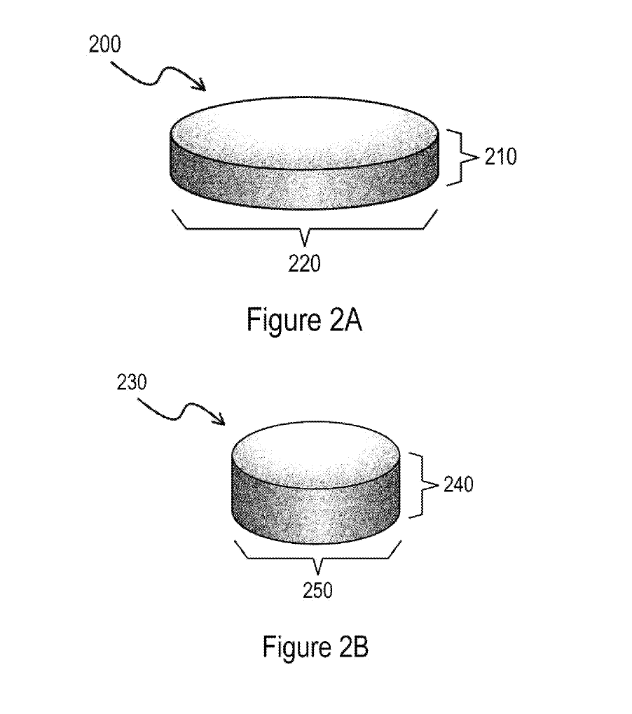

FIG. 1A illustrates one embodiment of a generally cubic plate silver nanoparticle 100 that has a radius of curvature at its corners defined by the circle 110. Under certain environmental conditions a shape change can occur and in some embodiments this can result in an increased radius of curvature at the corners of the nanoparticle. FIG. 1B illustrates one embodiment of a generally cubic plate silver nanoparticle 120 that has an increased radius of curvature 130 when compared to the radius of curvature 110. FIG. 2A illustrates one embodiment of a generally plate shaped nanoparticle 200 with a thickness 210 and a width 220. In an embodiment, under certain environmental conditions the shape of the plate shaped nanoparticle 200 can change shape into another particle 230, illustrated in FIG. 2B that has an increased thickness 240 and a decreased edge length (e.g., width) 250.

In an embodiment the degree to which the particles are metastable is controlled by the particular crystal facets that the nanoparticle expresses. Different crystal facets have different degrees of lability of silver ion associated with them. By controlling the facets that are expressed on the nanoparticle, the off rate of silver ions from the silver nanoparticle surface can be controlled.

In an embodiment the silver nanoparticles can have a pyramidal shape and an oxidation process generating silver ions that leads to an increase in the radius of curvature of the vertex between one or more crystal faces.

In an embodiment the silver nanoparticles can have a cubic shape and on exposure to moisture undergo an oxidation process releasing silver ions, leading to an increase in the radius of curvature of the vertex between one or more crystal faces.

Ion Release from Metastable Silver Nanoparticles

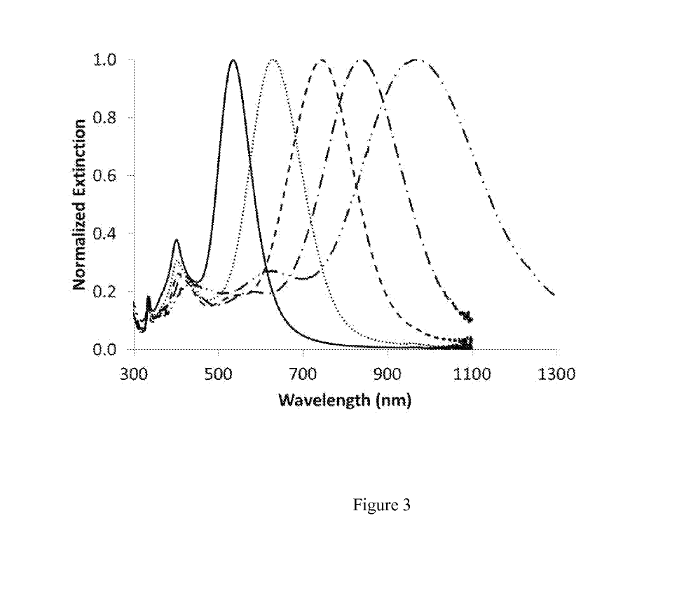

In an embodiment, the change in the shape of silver nanoparticles modifies the optical properties of the silver nanoparticles. Silver nanoparticles can support surface plasmon modes and are referred to as plasmon resonant particles. FIG. 3 illustrates the optical spectra of one embodiment of silver nanoplates that have different aspect ratios. Each of these particles in solution has a different color that is discernible by the eye. In one embodiment, the shape of the nanoparticles will change due to ion dissolution from the surface of the nanoparticle where the silver ion dissolution rate is approximately the same at all points on the surface of the nanoparticle. This results in the size of the particle being reduced. In oneembodiment, the ion dissolution rate from the surface of the nanoparticle is not the same at all points on the surface. For example, the ion release rate from the edges of a plate shape nanoparticle may be greater than the ion release rate from the surface of the particle. In this case, the shape change of the particle is due to a change in the aspect ratio of the particle. In one embodiment, the silver ions that are released from the surface either stay in solution or complex with other chemicals or surfaces. In one embodiment, the silver ions that are released from the surface can rebind to the same silver nanoparticle or to other silver nanoparticles in the composite. The rebinding of the silver ions to the silver nanoparticles can be uniform on all silver surfaces or can preferentially bind to one or more faces of the silver nanoparticles. In one embodiment, the silver ion release rate and the silver ion deposition rate is a function of the size of the particle. For example, the silver ion release rate can be greater for smaller particles than for larger particles. In one embodiment, the free silver ions in solution form new silver nanoparticles. When new silver nanoparticles are formed they are generally spherical and the shape distribution of the nanoparticles on the substrate or in solution can be different than the original shape distribution.

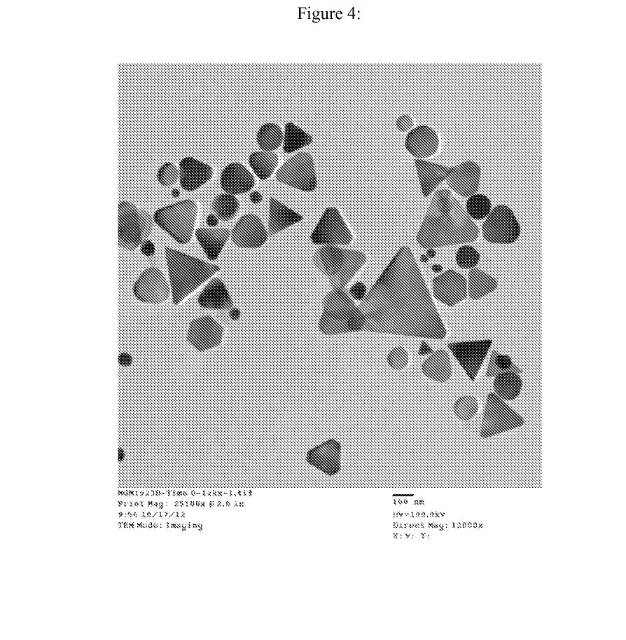

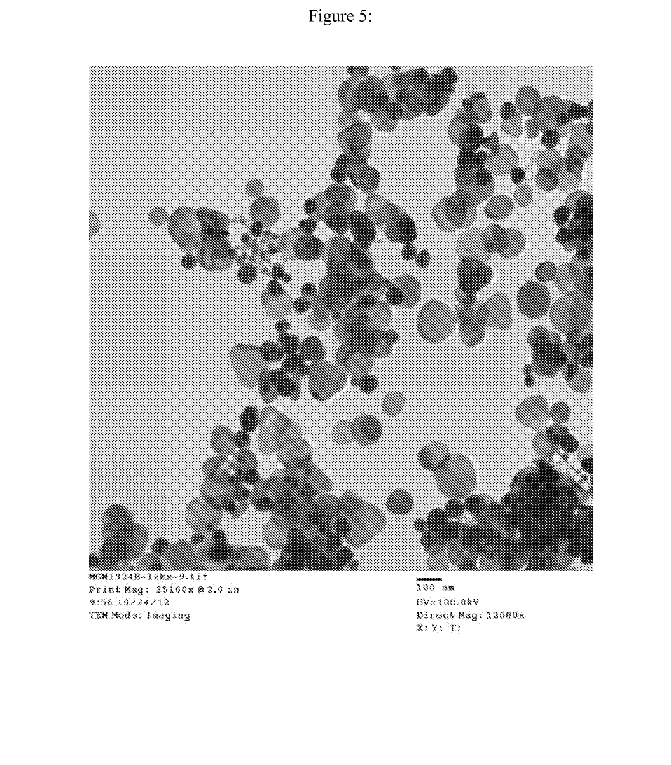

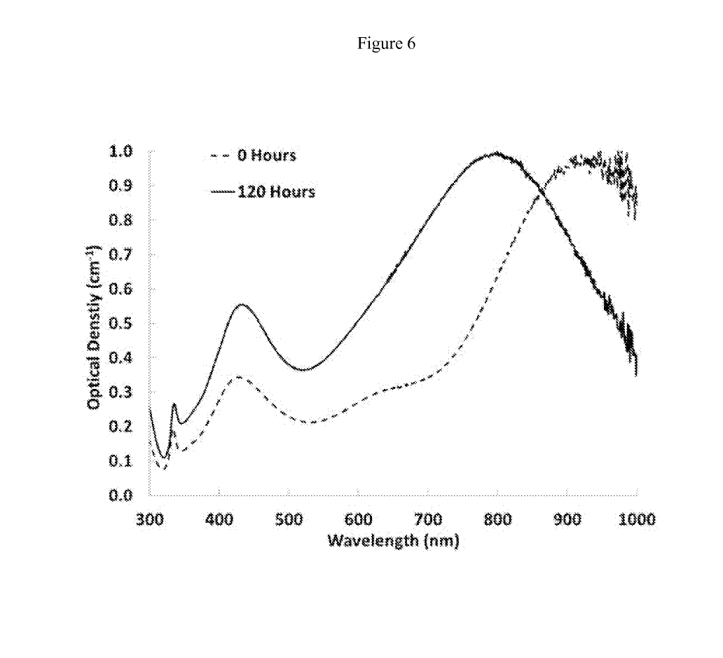

FIG. 4 illustrates transmission electron microscopy (TEM) images of some embodiments of silver nanoplates immediately after synthesis. FIG. 5 illustrates a TEM image of one embodiment of silver nanoparticles that were stored in an open container for 5 days. FIG. 6 shows the UV Visible spectrum of the one embodiment of particles that have changed shape over time. The ratio of spheres to disks to triangles was 18:28:53 for the TEM sample in FIG. 4 (time 0) and 38:47:16 for the TEM sample in FIG. 5 (time 5 days). The average diameter of the spheres, disks, and triangles was 55 nm, 130 nm, and 170 nm, respectively for the TEM sample in FIG. 4 (time 0). The average diameter of the spheres, disks, and triangles was 61 nm, 116 nm, and 137 nm, respectively in FIG. 5 (time 5 days). This data demonstrates that both the distribution of shapes and the sizes is changing with time. The peak extinction wavelength was initially 930 nm. Five days later, the peak extinction wavelength was 790 nm. The shape change induced a peak extinction wavelength shift of 140 nm. In some embodiments, a peak wavelength shift of at least 5 nm, 10 nm, 20 nm, or 50 nm constitutes a perceptible shift in the color of the particles.

In one embodiment, the visible color shift that is associated with the change in the shape of the metastable particles provides information on the state of the silver nanoparticles. The color change of the silver nanoparticles is associated with the shape of the particle which in turn is a function of the silver ion release rate and the silver ion deposition rate on the silver nanoparticles. The end user of the composite can utilize both the color intensity (measuring how much is loaded onto the composite) and the color wavelength (the current shape of the particle) to determine the state of the silver nanoparticles in the composite. In one embodiment, the color can be used to determine whether the composite is still efficacious for wound treatment. In one embodiment, the color can be used to determine whether or not a washing step removed or altered the silver nanoparticles in the composite.

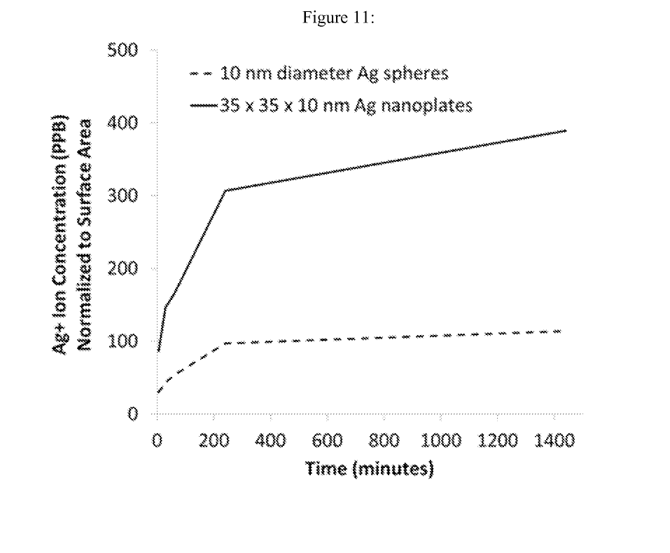

In one embodiment, a silver nanoparticle or silver nanoplate with vertices, corners, or edges of high curvature, when contacted with a solvent, releases silver ions at an enhanced rate relative to a composition of silver nanoparticles without high curvature having about the same or more exposed surface area of silver. Silver ion release as a function of time of nanospheres and nanoplates is shown in FIG. 11. The silver ion release data is normalized for equivalent surface area. The high curvature at the edges of the silver nanoplates contributes to accelerated ion release and results in approximately four (4) times more ions released from a given surface area on silver nanoplates vs. spherical nanoparticles. In some embodiments the silver nanoparticle of the present invention has at least one vertex, corner or edge with a radius of curvature that is at least four (4) times smaller than the longest dimension of the silver nanoparticle, In other embodiments the silver nanoparticle has at least on vertex, corner, or edge with a radius of curvature that is at least 5, at least 6, at least 8, at least 10, at least 20, at least 50, at least 100, or at least 500 times smaller than the longest dimension of the silver nanoplate.

In an embodiment, the degree to which the particles are metastable is controlled by the environment. In some embodiments the medium surrounding the silver nanoparticles is a gas which can include gases such as air or an inert atmosphere. In some embodiments the environment is a full or partial vacuum. In an embodiment, the metastable nanoparticles can undergo a chemical change associated with the long term storage in the gas environment. This change can include the oxidation of the silver or the binding of aerosolized molecular species to the surface of the silver including molecules that contain amines or mercapto components. In one embodiment the medium is moist. A moist environment is defined to be wet, slightly wet, damp, or humid. In the case where the moist environment is a liquid, the liquid can be a pure liquid or any combination of liquids. In a preferred embodiment, the liquid media consists of a substantial portion of water and is referred to as an aqueous medium. The liquid media can also contain a percentage of chemical or biological solids. In one embodiment the aqueous medium is a biological fluid such as a wound exudate, blood, or serum. In some embodiments, the moist environment creates a liquid layer near the surface of the silver nanoparticles. In this embodiment, silver ions can diffuse from the surface of the nanoparticles into solution. In an embodiment, the Ag⁰ of the metal nanoparticles is oxidized into soluble Ag⁺¹ ions. Free silver ions in solution can remain in solution, bind to another entity in contact with the solution, or be reduced back to Ag⁰ on the surface of the silver nanoparticles or somewhere else.

### Stabilized Silver Nanoplates

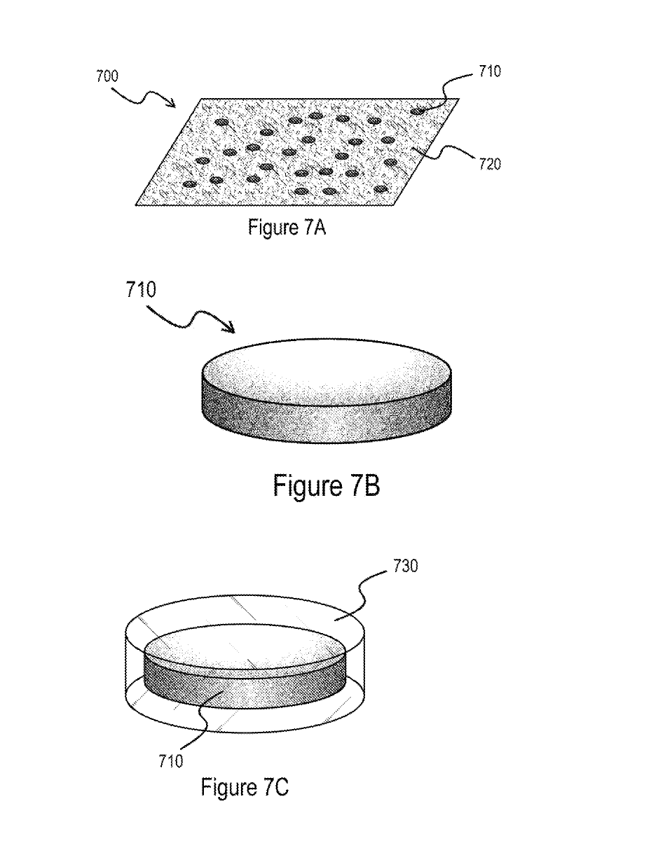

In an embodiment, a composite includes silver nanoplates with a stability modulant to form stabilized silver nanoplates. A stability modulant is any material that affects the stability of the metastable nanoparticles. In one embodiment the stability modulant is a coating on the nanoparticle that increases the stability of the metastable nanoparticles. In one embodiment the stability and metastable nanoparticle form a stabilized silver nanoplate that, when contacted with a solvent, releases silver ions at a reduced rate relative to a silver nanoplate without a stability modulant (non-stabilized silver nanoplate). FIG. 7A illustrates a composite 700 that consists of silver nanoparticles 710 and a substrate 720. In one embodiment, the silver nanoparticles are coated with an encapsulant 730 illustrated in FIG. 7C. Nanoparticles coated with a stabilant can retain their shape for days, weeks, months or years in either or both wet or dry environments. The stabilant can be a chemical or biological agent that is physibsorbed to the surface, molecularly bound to the surface through specific interactions (e.g. thiol or amine), or encapsulate the surface (i.e. a metal oxide or metalloid oxide shell). In various embodiments, examples of chemical agents that can be bound to the surface of the silver nanoparticles include citric acid, polysulphonates, vinyl polymers, alkane thiols, dithiols, carbohydrates, ethylene oxides, phenols, carbohydrates, organic ligands, stimulus responsive polymers, polyacetylene, sodium borate, sodium bicarbonate, sodium ascorbate, chlorine salts, a primary amine or a secondary amine. In some embodiments the silver nanoparticles are coated with poly(sodium) styrene sulfonate, polyvinyl alcohol, polyvinyl pyrrolidone, tannic acid, lipoic acid, dextran, and polyethylene glycol (PEG) including PEG molecules which contain one or more chemical groups (e g amine, thiol, acrylate, alkyne, maleimide, silane, salts (e.g. sodium borate or sodium bicarbonate), azide, hydroxyl, lipid, disulfide, fluorescent molecule, or biomolecule moieties). In some embodiments the amount of the coating is titrated into the solution of silver nanoparticles so that there is less than 1%, 5%, 10%, 20%, 30%, 40%, 50%, 75% or 100% coverage of the surface of the silver nanoparticle with the coating. In other embodiments, the coating encapsulates the silver nanoparticle in one or more layers. In some embodiments, the coating molecule modulates the release rate of silver ions from the surface and has an effect on the rate of change of the indicator color. In a preferred embodiment, a thiolated molecule such as mercapto-PEG or lipoic acid is used in sub-monolayer coverage to modulate the rate of color change of the silver particles (e.g. a silver nanoplate) in solution or embedded into a composite. In various embodiments, specific biomolecules of interest include proteins, peptides, and oligonucleotides, including biotin, bovine serum albumin, streptavidin, neutravidin, wheat germ agglutinin, naturally occurring and synthetic oligonucleotides and peptides, including synthetic oligonucleotides which have one or more chemical functionalities (e g amine, thiol, dithiol, acrylic phosphoramidite, azide, digoxigenin, alkynes, or biomolecule moieties). Specific encapsulating chemical agents of interest include metal oxide shells such as SiO₂ (silica oxide) and TiO₂ (titanium oxide). Stabilizing agents may be added prior to the formation of silver nanoparticles, during the formation of silver nanoparticles, or after the formation of silver nanoparticles. The thickness of the coating can be a monolayer or sub-monolayer or a shell that fully or partially encapsulates the nanoparticle. In one embodiment, a partial encapsulation means that the nanoparticle is at least about 10% covered by the shell, such as 20, 30, 40, 50, 60, 70, 80, 90, 95, 99, 99.9% or greater than 99.9% covered, and the covered or uncovered region(s) may be contiguous or discontiguous. In various embodiments, the thickness of the shell can range from 0.1 nm to 100 nm, such as 0.5, 1, 5, 10, 15, 20, 25, 30, 35, 40, 45, 50, 55, 60, 65, 70, 75, 80, 85, 90, 95 or 100 nm. The thickness of the shell can range from 1 nm to 100 nm. In some embodiments the shell is porous (e.g. silica).

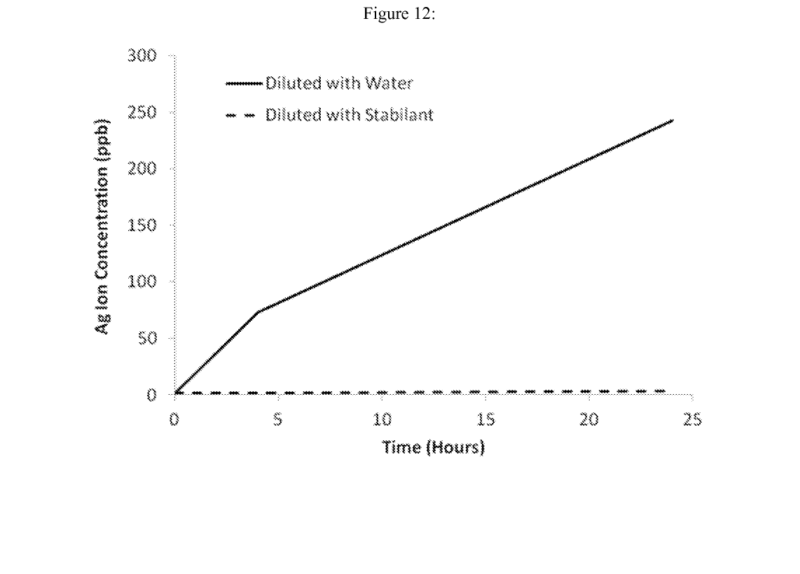

In one embodiment a composition of stabilized silver nanoplates comprise a combination of a polyvinyl polymer and a salt including borate salt or a bicarbonate salt. FIG. 12 shows the ion release from concentrated silver nanoplates stabilized with borate and a polyvinyl polymer (PVP) diluted 200-fold with water or diluted 200-fold with 5 mM borate. In contact with a solvent in the absence of the borate stabilant, the silver nanoplates rapidly release silver ions whereas in the presence of the both PVP and borate stabilant, the silver nanoplates retain their shape and do not appreciably release silver ions even at 200 fold reduced concentration. In an embodiment, the metastable silver nanoparticles are combined with one or more stability modulants into a paste, cream, or liquid. In one embodiment the metastable silver nanoparticles are coated with a protectant. In one embodiment, the suspension medium contains an etchant. In one embodiment, a combination of etchants and protectants are combined with the silver nanoparticles into the suspension medium.

In one embodiment, the stability modulant can affect the binding strength of the silver nanoparticle to the substrate. For example, proteases or other biological processes in a wound bed could accelerate the release rate of the silver nanoparticle from the substrate into the local environment. In one embodiment, the stability modulant is an acid, solvent, or other biological or chemical entity that can interact with the binding forces adhering a silver nanoparticle to the substrate.

In various embodiments, metallic silver nanoparticles, on exposure to air and water, can undergo oxidation to generate silver ions. The extent and the nature of this oxidation depends on the environment of the silver and the shape of the silver nanoparticles. In one embodiment, the nanoparticles are shelled with a layer that modulates access of the oxidizing species to the surface which controls the rate at which the silver ionizes. In one embodiment, the stability modulant protects the silver nanoparticles from thiols. In an embodiment the use of a layer of oxide such as silica, or a layer of polymer such as polystyrene on the surface of the silver nanoparticles, can control the rate of generation of silver ions from the surface.

In one embodiment, the use of a reductant on the surface of the silver nanoparticles can reduce the oxidation of the silver on the silver nanoparticle. In one embodiment, the reductant on the surface of the silver is fully or partially removed from the surface when the silver nanoparticles is exposed to moisture. In one embodiment the reductant is in the form of an ascorbate, citrate or other organic or inorganic reductant and is closely associated with the surface of the silver metal nanoparticles until dissolved away with moisture. In one embodiment the reductant stays in close proximity to the silver and reduces the off rate of silver ions from the surface regardless of the moisture conditions.

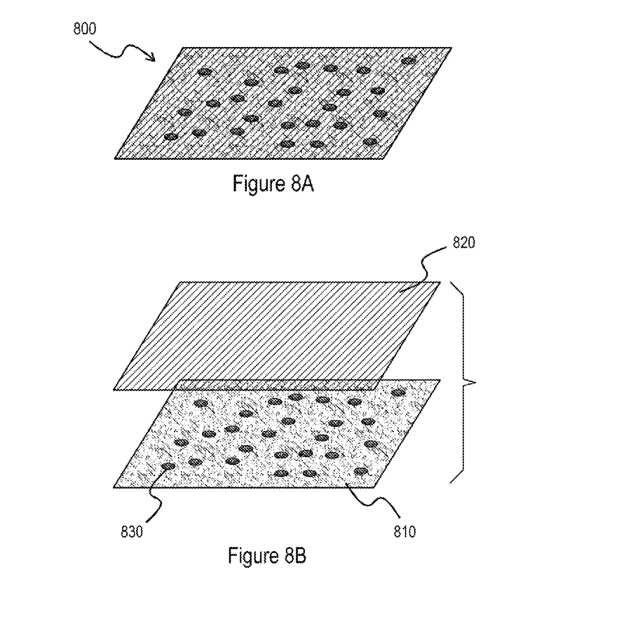

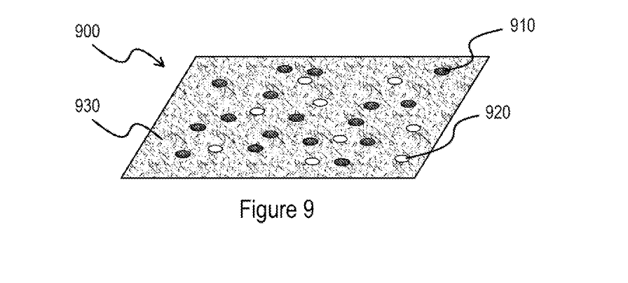

In one embodiment, there is a stability modulant in the composite that is a material that accelerates the dissolution of the metastable silver nanoparticles. In one embodiment, the stabilant modulant is added to the composite as a coating. FIG. 8A illustrates an embodiment of a composite 800 that consists of a substrate, silver nanoparticles and a coating. FIG. 8B illustrates the components of the composite 800. The coating 820 is applied to the substrate 810 which contains silver nanoparticles 830. The stabilant modulant is dissolved when the composite comes in contact with moisture which affects the properties of the liquid that is contact with the composite (the environment). In some embodiments the stabilant modulants either raises or lowers the pH of the environment, contains molecules that can displace or dissolve surface coatings or shells on the silver particles, contains amines, contains thiols, contains oxidants, contains salts, contains etchants, or contains halides. In some embodiments, the stabilant modulant coating rapidly dissolves. In other embodiments, the stabilant modulant coating is mixed with other compounds that slow the release of the stabilant modulant allowing the modulant to be released over a period of hours, days, weeks, or months. In one embodiment the stabilant modulant is a population of particles that are bound to the substrate. FIG. 9 illustrates a composite 900 that consists of silver nanoparticles 910 and stability modulant particles 920 that are attached to a substrate 930. In one embodiment the particles can dissolve with time to release stabilant modulant molecules that accelerate the dissolution of the silver nanoparticles. The particles can be made from a single stabilant modulant, a combination of stabilant modulants, or can include other chemicals and the stabilant modulant. The other chemicals present in the particle can include slow release compounds such as PLGA.

In an embodiment, an oxidant can be employed to increase the silver ion off rate from the particles. This can include any species likely to oxidize silver and the oxidant can stem from the environment, the composite it is placed in or can be a part of the composite itself. Example oxidants include but are not limited to amines, thiols, other metal salts or oxidizing organic species.

In an embodiment, a combination of oxidant and reductant can be employed in the composite to modulate the rate and amount of silver ion dissolution. In a particular embodiment the reductant is associated with the surface of the silver nanoparticles, preventing generation of the ions until it is desired to do so. In one embodiment the oxidant is spatially displaced from the surface of the silver nanoparticles and it water soluble. On exposure to moisture, the reductant is displaced from the surface of the silver nanoparticles and the surface is exposed to an oxidant which has diffused to the surface consequently increasing the rate of dissolution of the silver nanoparticles on exposure to moisture.

In some embodiments, the composite includes a coating that increases the stability of the silver nanoparticles during dry storage and additional stability modulants in the composite that accelerate the dissolution of the silver nanoparticles when exposed to moisture. In some embodiments, the composite is stable for long periods of time when not in use and stored in a wide variety of temperature and humidity environments while retaining the ability to release silver ions when in a moist environment. In one embodiment the coating on the particles is a porous shell (e.g. silica). In other embodiments, the coating on the particle increases the binding strength to the substrate.

### Medical Devices

In some embodiments a medical device is provided comprising a surface for application to a human subject, wherein the surface comprises a plurality of stabilized encapsulated silver nanoplates. In various embodiments, medical devices include tube, syringe, bandage, sheet, sock, sleeve, wrap, shirt, pant, mesh, cloth, sponge, paper adhesive, catheter, orthopedic pin, plate, implant, tracheal tube, insulin pump, wound closure, drain, shunt, dressing, connector, prosthetic device, pacemaker lead, needle, dental prostheses, ventilator tube, ventilator filter, pluerodesis device, surgical instrument, wound dressing, incontinence pad, sterile packaging, clothing, footwear, diaper, sanitary pad, biomedical/biotechnical laboratory equipment, table, enclosure, and/or wall covering.

In some embodiments the medical device is an IV administration set or component thereof, IV extension set, IV connector, IV bag, and the like. Generally, the stabilized encapsulated silver nanoplates can be incorporated in/on the lumen of IV tubing or in/on a substrate that sits within a plastic housing along the IV line such as a silicone ring or fitting. Components in which a composite containing silver nanoplates may be housed include: Drip chambers, luer access valves, male luers, stopcocks, slide chambers, labels attached to the IV set, piggy back ports, piggy back sets or secondary sets, Y injection sites, end caps, and the like.

In some embodiments stabilized encapsulated silver nanoplates are provided on or within the eluting portion of a drainage catheter device comprising: an elongate flexible tube body including a distal length configured to indwell a patient; and a body lumen extending longitudinally through at least a lengthwise portion of the distal length, the lumen substantially defined by an inner diameter surface of the tube body; the distal length including at least one aperture disposed through a wall of the body, and in fluid communication with the body lumen; wherein at least one portion of the distal length is configured as an eluting portion that includes at least one surface constructed to elute a sclerotic agent; and wherein at least one structure is provided and configured to decrease a probability of direct contact between the eluting portion and a surface external of the device disposed immediately adjacent the eluting portion, for example the device described in U.S. Pat. Pub. No 2012/036898, which is incorporated by reference in its entirety herein.

In some embodiments stabilized encapsulated silver nanoplates are provided in patient ventilation device. In an embodiment stabilized encapsulated silver nanoplates are imbued in all or a portion of facial skin interface, compliant nose bridge seal, micro-grooves, porous material, and/or wicking material, for example the device described in U.S. Pat. Pub. No. 2012/037163, which is incorporated by reference in its entirety herein.

### Surfaces or Substrates

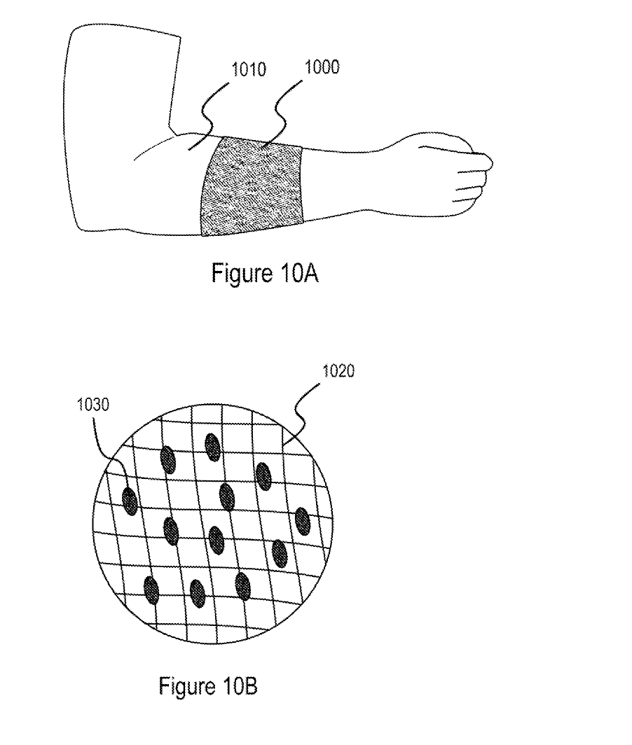

In one embodiment of the invention, the metastable silver nanoparticles (e.g., including stabilized encapsulated silver nanoplates) are associated with a substrate or a surface. Examples of substrates or surfaces include non-woven fibers, woven fibers, natural fibers, fibers from animals (e.g. wool, silk), plant (e.g. cotton, flax, jute), mineral fibers (e.g. glass fiber), synthetic fibers (nylon, polyester, acrylic), cloth, mesh, bandages, socks, wraps, other articles of clothing, sponges, high porosity substrates, particles with diameters greater than 1 micron, beads, hair, skin, paper, absorbant polymers, foam, wood, cork, slides, roughened surfaces, biocompatible substrates, filters, silicone, hydrogels, plastics, medical grade plastics or polymers, or medical implants. FIG. 10A illustrates a bandage 1000 that is applied to an arm (1010). FIG. 10B shows a close-up of the structure of the bandage 1000. The substrate is a cloth of woven or otherwise combined fiber 1020 that has silver nanoparticles 1030 bound to the surface of the fiber.

Provided in one embodiment is an article comprising a material suitable for incorporation into a medical device or article of manufacture, the material comprising a surface wherein a plurality of stabilized encapsulated silver nanoplates are disposed substantially on and/or in the surface at a concentration sufficient to provide an anti-microbial activity and/or colorimetric indication when activated by a solvent. In some embodiments the surface comprises a metal surface, plastic surface, silicone, fiber surface including a porous network of fibers, or a glass surface. In some embodiments the surface comprises a synthetic bioabsorbable polymer, for example: polyesters/polylactones such as polymers of polyglycolic acid, glycolide, lactic acid, lactide, dioxanone, trimethylene carbonate etc., polyanhydrides, polyesteramides, polyortheoesters, polyphosphazenes, and copolymers of these and related polymers or monomers. In some embodiments a silicone surface is provided comprised of polydimethylsiloxane, cross-linked polydimethylsiloxane, silicate resin in polydimethylsiloxane, silica in polydimethylsiloxane, organofunctional siloxane, silicone polyether, silicone alkyl wax or other functionalized silicones.

In some embodiments the surface comprises a naturally derived bioabsorbable polymer including proteins: albumin, fibrin, collagen, elastin; polysaccharides: chitosan, alginates, hyaluronic acid; and biosynthetic polyesters: 3-hydroxybutyrate polymers.

Encapsulated silver nanoplates and bioabsorbable polymers forming a antimicrobial composition and or colorimetric indicator are useful for wound closure: including for example sutures, staples, and adhesives; tissue Repair: including for example meshes for hernia repair; prosthetic devices: including for example internal bone fixation, physical barrier for guided bone regeneration; tissue engineering: including for example blood vessels, skin, bone, cartilage, and liver; controlled drug delivery systems: including for example microcapsules and ion-exchange resins; and wound coverings or fillers: including for example alginate dressings and chitosan powders. In some embodiments the surface is inert and/or substation substantially anhydrous prior to use of the medical device.

### Surface Loading

In some embodiments stabilized encapsulated silver nanoplates are substantially localized on a surface. Encapsulated silver nanoplates may partially or fully coat the surface and can have a surface coating that is a partial layer, a fully formed layer or a multi-layer on the surface. The average thickness of the silver nanoplate layer can range from 2 nm to 100 nm, 2 nm to 500 nm, 10 nm to 500 nm or from 10 nm to 1000 nm, or from 10 nm to 3000 nm. In various embodiments, encapsulated silver nanoplates silver are present on the surface at a surface density of 0.0001 mg to 1 mg per square inch (e.g., including from 0.0-1 mg to 1 mg, 0.001 mg to 0.1 mg, 0.001 mg to 1 mg, 0.01 mg to 1 mg, 0.01 mg to 10 mg and/or 0.001 mg to 10 mg, or any ranges therein)). Encapsulated silver nanoplates may be retained on a surface by adsorption or by adhesion such that they are physisorbed, covalently bonded, or electrostatically bound to the surface.

In some embodiments stabilized encapsulated silver nanoplates are substantially disposed in a surface. In some embodiments they may be disposed in the surface when the surface is produced. In various embodiments, encapsulated silver nanoplates silver are disposed in the surface at a surface density of 0.0001 mg to 1 mg per square inch (e.g., including from 0.001 mg to 1 mg, 0.001 mg to 0.1 mg, 0.001 mg to 1 mg, 0.01 mg to 1 mg, 0.01 mg to 10 mg and/or 0.001 mg to 10 mg, or any ranges therein).

In one embodiment, the high optical density solutions of silver nanoparticles at a concentration of at least 0.1 mg/mL, 1 mg/mL, 10 mg/mL, 100 mg/mL (e.g., 1 to 10, 3 to 30, 5 to 50, 10 to 20, 5 to 50, 3 to 50, 1 to 100 mg/mL, 10 to 100, 20 to 100, 30 to 100 mg/mL) are incubated with the substrate. In one embodiment, the high optical density solutions of silver nanoparticles at a concentration of at least 1 mg/mL, 10 mg/mL, or 100 mg/mL are incubated with the substrate. In one embodiment the silver nanoparticles are prepared at an optical density of at least 10, 100, 300, 500, 1000, or 2000 cm⁻¹ at their peak resonant wavelength before incubating with the substrate.

In one embodiment the substrate is chemically treated to increase the binding of the silver nanoparticles to the substrate. For example, the substrate could be functionalized with a molecule that yielded a positively or negatively charged surface. In one embodiment, the pH of the incubating solution is selected in order to optimize binding. In one embodiment, the silver nanoparticles cover at least 5%, 10%, 20%, 30%, 50% or 75% of the substrate. In one embodiment, other solvents or chemicals are added to the incubation solution. In one embodiment a biological linker (e.g. antibodies, peptides, DNA) is used to bind the high optical density silver nanoparticles to the surface of the substrate. In one embodiment the substrate is chemically modified to have a higher affinity to the silver nanoparticles. In a particular embodiment a protein based substrate in which dithiol bridges are present is reduced, generating free thiols that can bind to the surface of the silver nanoparticle. In one embodiment, the incubation is for less than 1 minute, 5 minutes, 20 minutes, 60 minutes, 120 minutes, or greater than 120 minutes. In one embodiment the silver nanoparticles are physisorbed, covalently bounded, or electrostatically bound to the substrate. In one embodiment, the faces of the high aspect ratio particles that have the largest surface area preferential bind to the substrate. In one embodiment, silver nanoparticles with a high aspect ratio shape bind with more force to the substrate than silver nanoparticles with a lower aspect ratio.

In some embodiments stabilized encapsulated silver nanoplates are disposed in a substrate including a plastic, silicone, glass, hydrogel,or other polymer. Generally, shape change of stabilized encapsulated silver nanopiates can be slowed by embedding silver nanoplates in a substrate to limit diffusion of silver ions out of the substrate and/or the diffusion of solvents or etchants into the substrate. Diffusion of ions and solvents through the material is a function of the substrate material properties and substrate thickness. Generally, water permeable or porous substrates such as hydrogels or bioabsorbable polymers provide faster diffusion of silver ions, solvents, and etchants than silicones or plastics. Thus, a silicone, plastic, or alternative substrate that limits diffusion/permeability of water or other solvents/etchants is a preferred embodiment to slow the shape change of an encapsulated silver nanoplate from hours to days to weeks or longer. Furthermore, the thickness of the substrate can be varied from 100 nm to 10 mm or larger to control the rate of shape change of stabilized encapsulated silver nanoplates to provide a desired silver ion release or colorimetric signal. In some embodiments the substrate thickness is from 100 nm to 1000 nm, 300 nm to 3 microns, 1000 nm to 10 microns, 3 microns to 30 microns, 10 microns to 100 microns, 30 microns to 300 microns, 100 microns to 1 mm, 300 microns to 3 mm, 1 mm to 1 cm, 3 mm to 3 cm, 1 cm to 10 cm or greater. In a preferred embodiment, the stabilized silver nanoplates are embedded in a silicone substrate that is between 1 micron and 1 mm in thickness to extend the shape change of the stabilized encapsulated silver nanoplates after exposure to moisture or etchant from occurring over 1 day to occurring over 1 week or longer. In a preferred embodiment the stabilized encapsulated silver nanoplates are encapsulated with a metal oxide coating including silica. to provide a compatible surface for integration into different composites, to modulate shape change rate and as a. template for etching to occur.

### Color Detection/Colorimetric Signaling

In one embodiment, the detectable (e.g., visible) color shift that is associated with the change in the shape of the metastable particles provides information on a state of the silver nanoparticles. For example, the color change of the silver nanoparticles is associated with the shape of the particle, which in turn is a function of the starting shape, the silver ion release rate, the silver ion deposition rate on the silver nanoparticles, and encapsulations that direct etching to maintain plasmonic shape. Thus, stabilized encapsulated silver nanoplates can display a visibly detectable color shift when activated by a solvent. The end user of the composite can utilize both the color intensity (measuring how much is loaded onto the composite) and/or the color wavelength (the current shape of the particle) to determine the state of the silver nanoparticles in the composite. In one embodiment, the color can be used to determine whether the composite is still efficacious for wound treatment. In one embodiment, the color can be used to determine whether or not a washing step removed or altered the silver nanoparticles in the composite.

In some embodiments, an article is provided comprising silver nanoplates with high curvature such that the article demonstrates by a detectable change in color the activation of silver ion release as the ‘plasmonic’ structure of the silver nanoparticle changes. The color change may not happen instantly, but is detectable after several minutes, hours, or days. In some embodiments colorimetric signaling is provided in the visible spectrum so that a consumer can visually monitor the ion release state of the article. In some embodiments colorimetric signaling is provided by a spectral imaging/detection system. In some embodiments a color legend is provided with the article with instructions for use to determine the ion release state. In one embodiment silver nanoplates of specific dimensions are provided with a plasmonic resonance structure such that the article color shifts from blue to violet to red to orange to yellow as ions release and the shape of the particle changes (FIG. 1). In one embodiment silver nanoplates of dimensions about 20-60 nm, 20 to 100 nm or 30-150 nm in the long axis and 5-20 nm in the short axis are provided such that the article color shifts from blue to violet to red/orange to yellow during ion release and may eventually turn clear on full dissolution of particles from ion release. In one embodiment the plasmonic shape creates an absorbance and reflection pattern in such a way to color a solution, gel, semi-solid, solid, or a surface blue in the initial state. Colorimetric signaling may be provided as a shift in hue or color intensity or both. Colorimetric signaling may be provided as a result of shape changes of silver nanoplates, full dissolution of silver nanoplates, or diffusion of silver nanoplates away from the article.

In some embodiments colorimetric signaling is provided as a detectable shift of the surface plasmon peak wavelength of silver nanoparticles from between about 500 nm and about 700 nm to between about 300 nm and 500 nm; from between about 650 nm and about 1100 nm to between about 300 nm and about 650 nm; from between about 600 nm and about 650 nm to between about 300 nm and about 600 nm; from between about 550 nm and about 600 nm to between about 300 nm and about 550 nm; from between about 500 nm and about 550 nm to between about 300 nm and about 500 nm; or from between about 450 nm and about 500 nm to between about 300 nm and about 450 nm. In some embodiments colorimetric signaling is provided as a detectable shift of the surface plasmon peak wavelength of silver nanoparticles from a higher wavelength to a lower wavelength. In some embodiments the peak extinction wavelength shift comprises a shift or shortening of at least 5 nm, at least lOnm, at least 50 nm, at least 100 nm, at least 200 nm, at least 300 nm, at least 400 nm, at least 500 nm, at least 600 nm, or more than 600 nm. In some embodiments colorimetric signaling is provided wherein the signaling comprises a detectable decrease in absorbance of light having a wavelength of between about 500 nm and about 700 nm and an increase in absorbance of light having a wavelength of between about 300 nm and about 500 nm; a detectable decrease in absorbance of light having a wavelength of between about 650 nm and about 1100 nm and an increase in absorbance of light having a wavelength of between about 300 nm and about 650 nm; a detectable decrease in absorbance of light having a wavelength of between about 600 nm and about 650 nm and an increase in absorbance of light having a wavelength of between about 300 nm and about 600 nm; a detectable decrease in absorbance of light having a wavelength of between about 550 nm and about 600 nm and an increase in absorbance of light having a wavelength of between about 300 nm and about 550 nm; a detectable decrease in absorbance of light having a wavelength of between about 500 nm and about 550 nm and an increase in absorbance of light having a wavelength of between about 300 nm and about 500 nm; or a detectable decrease in absorbance of light having a wavelength of between about 450 nm and about 500 nm and an increase in absorbance of light having a wavelength of between about 300 nm and about 450 nm.

Colorimetric signaling can drive safe and appropriate product use by consumers (e.g. reapplying bandages, replacing spent filters, replacing cleaning or sanitizing agents). In some embodiments, high curvature silver nanoplates are provided in an article in a stable form with low ion release until activated to release ions by dilution with a solvent (water, ethanol, electrolytes, body fluids). In some embodiments activation is achieved by diffusion of stabilizing agents (e.g. borate salt) away from the article during use. Examples of antimicrobial products with colorimetric signaling include but are not limited to: filtration devices with anti-fouling or antimicrobial properties, wound dressings, band-aids, topical ointments, hand-sanitizers, water purifying tablets, gels, or fluids, laundering agent, fabric softeners, deodorant, sheets, towels, mats, socks, gloves, braces, air-purifiers, food storage containers, soaps, wraps, compression braces, underwear, tooth brushes, face masks, head wraps, cutting boards, hair irons, spray, pacifiers, refrigerators, vacuums, washing machines, computer mice, balm, bottles, wipes, and any of the articles or medical devices described herein.

Colorimetric signaling is useful alone in this invention with or without antimicrobial properties. Generally, colorimetric signaling can provide general indication or monitoring of the use of an article. In one embodiment, silver nanoplates are incorporated into an article to indicate the use time of the article (e.g., the time since activated or put in use). Use time is useful for indicating status, replacement, expiration and/or appropriate use of a perishable (e.g. food, milk, cheese, meats, other perishables), a medical device (e.g., IV sets, IV tubing, needless connectors, catheters, implantable devices, oral/nasal tubes or fittings, other medical devices), or other articles. In some embodiments use indication provides detection of an activation step or steps, such as moisture or salt exposure. Use indication may refer to the type or amount of exposure (e.g. salt concentration, pH) and/or the amount of time over which the exposure occurred. Some examples of use indicators include body moisture detector articles for ulcer prevention, strikethrough or leakage guards for wound dressings, detection displays in kits based on moisture, salt, or pH activation, and other articles where detection is useful.

In some embodiments the rate of dissolution of particles and color changes occurs such that at least 5%, 10%, 15%, 20%, 25%, 30%, 35%, 40%, 45%, 50%, 60%, 70%, 80%, 90%, 100% of silver mass is released in ion form in 1 second or less, from 1 to 10 seconds, from 1 second to 1 minute, from 1 second to 1 hour, from 1 minute to 1 hour, from 1 minute to 1 day, from 1 hour to 1 day, from 1 hour to 1 week, from 1 day to 1 week, from 1 day to 1 month, from 1 week to 1 month, from 1 week to 1 year, from 1 month to 1 year, from 1 month to 3 years, from 1 year to 3 years, or over greater than 1 year.

### Wound Dressings

In some embodiments, a wound dressing is provided comprising encapsulated silver nanoplates. Generally a wound dressing is comprised of at least two of three layers: a wound facing layer, an absorbent layer, and an outer layer. The encapsulated silver nanoplates localized on or disposed in the materials of one or more of the layers.

A) Wound Facing Layer

The first layer of the wound dressing is formed of a material, typically a perforated, preferably non-adherent material, which allows for fluids to penetrate or diffuse through in either or both directions. The perforated material may be formed of a woven or non-woven fabric such as cotton, gauze, a polymeric film such as polyethylene, nylon, polypropylene or polyester, an elastomer such as polyurethane or polybutadiene elastomers, or a foam such as open cell polyurethane foam. The first layer may also be an adherent material such as an adherent breathable or fluid permeable polymer that aids in fastening the dressing to the wound.

B) Absorbent Layer

The second, absorbent layer is formed from an absorbent material for absorbing moisture from the wound, or as in the case of a burn wound dressing, for holding moisture next to the wound. Preferably, the absorbent material is an absorbent needle punched non-woven rayon/polyester core. However, other suitable absorbent materials include woven or non-woven materials, non-woven being preferred made from fibers such as rayon, polyester, rayon/polyester, polyester/cotton, cotton and cellulose fibers. Exemplary are creped cellulose wadding, an air felt of air laid pulp fibers, cotton, gauze, and other known absorbent materials suitable for wound dressings. The absorbent layer may also contain polyurethane or polybutadiene elastomers, or a foam such as open cell polyurethane foam to aid in absorption and may be combined with the wound facing layer.

C) Outer Layer

The third layer of the wound dressing is optional, but is preferably included to regulate moisture loss, or to act as a barrier layer (for example for moisture, oxygen penetration), to carry an anti-microbial coating, or alternatively to act as an adhesive layer to anchor the wound dressing around the wound. In the case of burn wound dressings, the third layer is preferably formed of perforated, non-adherent material such as used in the first layer. This allows moisture penetration as sterile water and the like are added. This layer may also be useful to aid in detecting wound exudate strikethrough.

Encapsulated Silver nanoplates on Wound Dressings

The wound dressing of this invention preferably includes an anti-microbial material formed from encapsulated silver nanoplates. The material is applied to one or more of the layers but is most preferably applied at least to the first, wound facing layer to provide a localized anti-microbial effect next to the wound. The coating may also be applied to an additional layer, such as the outer layer, for additional anti-microbial effect or colorimetric signaling effect.

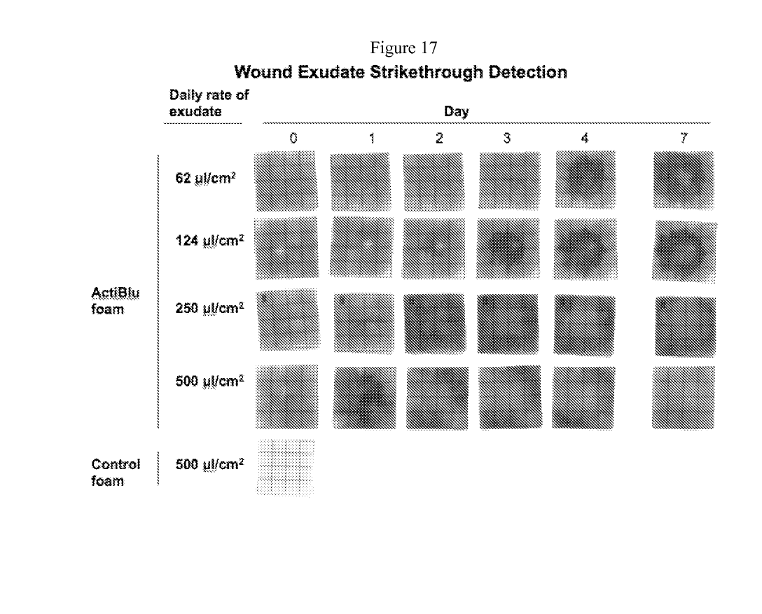

In one embodiment a foam dressing is provided comprising stabilized encapsulated silver nanoplates. Stabilized encapsulated silver nanoplates including silica coated silver nanoplates can be absorbed into the foam by soaking the foam in a solvent containing the nanoplates, such as water, and then evaporating the solvent away. Upon subsequent exposure to moisture, saline, or wound exudate, the silver nanoplates undergo shape changes, releasing silver ions for antimicrobial effect and visually indicating the degradation of their plasmonic shape. Generally, the rate of ion release and color change increases in the presence of saline or wound exudate as salts and proteins can act as etchants. The rate of ion release and color change also increases with higher rates of exudate production from the wound (FIG. 17). Thus, a foam with encapsulated silver nanoplates is useful for detecting exudate strikethrough, monitoring the rate of exudate production on a wound, and/or monitoring the status and expiration of antimicrobial effects in the wound dressing.

In other embodiments, stabilized encapsulated silver nanoplates are provided in hydrogel dressings, alginate dressings, hydrocolloid dressings, film dressings, hydrofiber dressings and the like. Generally, these dressing are formed by mixing encapsulated silver nanoplates into pre-polymer solutions during manufacturing to disperse them throughout the dressing. Alternatively, nanoparticles may be soaked into a dressing after manufacture. These dressings comprising stabilized encapsulated silver nanoplates are useful for detecting exudate strikethrough, monitoring the rate of exudate production on a wound, and monitoring the status and expiration of antimicrobial effects in the wound dressing.

### Burn Wound Treatment

For treatment of burns, moist dressings are preferable to potentiate wound healing and the antimicrobial effect of the silver materials. For example, the dressings are kept moist, at up to 100% relative humidity. Wound exudate may be sufficient in itself to maintain a desired humidity level. Otherwise, adding sterile water, for instance three times daily has been found to be sufficient. The wound dressing is thereafter wrapped in a known manner to keep the wound moist and clean. Dressings are changed as required for wound observation and cleaning, but need not be changed more frequently than every 24 hours, and can provide an anti-microbial effect for a much longer period of time.

In an alternative embodiment the encapsulated silver nanoplates are bound to a compression dressing that is applied directly to a wound. In one embodiment the compression dressing comprises one or more of wool, elastomers, nylon, cotton, or other natural or synthetic fibers. In an embodiment, the compression dressing contains one or more layers that absorbs and wicks moisture from the wound while releasing silver ions into the area of the wound. In one embodiment the compression dressing is shaped as a sock, a glove, or a tubular sleeve.

### IV Administration Sets and Connectors

Stabilized encapsulated silver nanoplates may be comprised in IV administration sets and connectors for both antimicrobial and indicator purposes. In a preferred embodiment, a continuous use indicator is formed to notify patients and caregivers of the status and/or expiration of an IV set or connector. Generally, IV administration sets, secondary sets, connectors, and other ports should be replaced no more frequently than 96 hours and no less frequently than 7 days. This practice has been shown to reduce infection rates caused by IV administration, improve patient safety, and save on costs. Stabilized encapsulated silver nanoplates may be embedded in or on a surfaces comprised of silicone, plastic, thin film or other substrates and disposed inside a plastic tubing or mold that allows constant flow of IV fluids over the surface. Salt from the IV fluids activates the etching of silver and shape changes in the plasmonic nanoparticles that generate the indicator color.

In some embodiments, the surface/substrate is silicone and the thickness of the silicone layer containing nanoplates is less than 0.01 mm, 0.03 mm, 0.1 mm, 0.3 mm, 1 mm or 3 mm. In some embodiments, the silicone also has an opaque or white pigment such as titanium oxide that causes only the outermost layer of encapsulated silver nanoplates to be visible. In some embodiments, a plastic housing fits around the silicone to cause saline or other IV fluids to flow over at least a portion of the silicone. In some embodiments, the concentration of the silver nanoplates in the silicone is from about 0.1 mg/cm³ to 2 mg/cm³ In some embodiments the silver nanoplates are encapsulated by silica or another coating with a thickness from 10-30 nm, 5-30 nm, 5-20 nm, 5-15 nm, 10-20 nm, or greater than 30 nm. In some embodiments, the silica is surface functionalized with different chemical agents, silane molecules, or other surface coatings to increase the compatibility of the coated nanoplates with the silicone. In some embodiments a legend is provided on or adjacent to the indicator demonstrating how the color of the nanoplate silicone corresponds to days of continuous use of the IV administration set or component (e.g., port, connector).

In one embodiment, the indicator is detatched from the IV flow circuit and attached on or near an external component of the IV set or connector. In this embodiment, color indication may be activated by breaking of a secondary container within or adjacent to the indicator unit to release water, saline, or other etchant over the substrate and activate the color change. Other embodiments might draw moisture from ambient air into the unit to activate exposure after a seal is broken. In some embodiments salt may be provided in the unit to mix with moisture from an external source. One in the art can appreciate that this external indicator may be useful in other medical and non-medical products as an indicator of duration of use or time since exposure (e.g. on or near a container used to hold food).

In one embodiment, a needless connector is provided with all or part of the silicone used in the connector comprising encapsulated silver nanoplates. In a preferred embodiment, the needleness connector is able to notify patients and caregivers of the status and/or expiration of antimicrobial activity and/or useful connector lifetime. Generally, the connector should have a flat, swabbable surface, positive pulse upon disconnect, and simple flow design visible fluid path. Exemplary connectors include MaxPlus and MaxGuard (Carefusion); Neutron, Microclave, Nanoclave, Bravo24, CLC200, Clave, and Antimicrobial Clave (ICU Medical); or other similar connector designs. In some embodiments only a portion of the silicone tip that sits in the lumen of the luer access valve comprises stabilized encapsulated silver nanoplates. In other embodiments the nanoplates are comprised in all of the silicone within the connector. In further embodiments, the stabilized encapsulated silver nanoplates may be comprised in the plastic casing that surrounds the silicone. In some embodiments the nanoplates may be sprayed or painted on the surface of the connector or a portion of the connector. In some embodiments a separate clear viewing window is provided so that a patient or caregiver may look at the status of the silicone color while the connector is engaged with a male luer lock.

In preferred embodiments the manufacturing of silicone components containing stabilized encapsulated silver nanoplates is achieved by mixing stabilized encapsulated silver nanoplates into pre-polymer solutions and injecting or depositing these solutions into pre-cast molds. In some embodiments, a silicone paint comprising nanoplates can be sprayed or painted on a component of the connector at any time in the manufacturing process. In other embodiments, nanoplates are mixed with a mold release compound and sprayed or painted onto a mold so as to only stick to the outermost portion of the silicone after setting. In some embodiments the silicone may be stamped in patterns including patterns of alternative encapsulated silver nanoplate forms.

Other useful IV administration set components in which stabilized encapsulated silver nanoplates may be comprised on or in include: Drip chamber, IV tubing, Luer Access valve, Male Luer. StopCock, Slide Chaber, Label, Piggy back port, Piggy back set (secondary set), Y injection site, and End caps.

Body Moisture Indicator/Ulcer Prevention

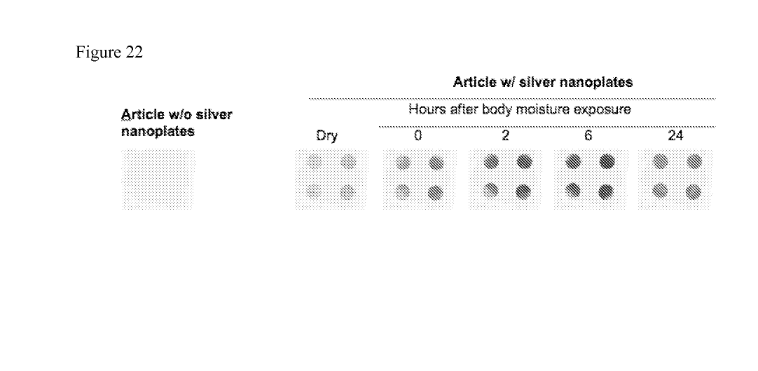

In some embodiments clothing, linens, or other articles are provided comprising stabilized encapsulated silver nanoplates that serve as body moisture indicators (e.g. indicator of exudate, sweat, incontinence). Body moisture detection is especially useful in the prevention of ulcers on immobile or minimally mobile patients (e.g. patients in wheelchairs or beds) Immobile patients are at risk of forming ulcers in areas exposed to pressure and moisture for long periods of time. Example articles for which body moisture detection is useful include diapers, undergarments, bed or seat covers, outer garments, socks, and other articles. Stabilized encapsulated silver nanoplates including silica coated silver nanoplates can be printed or absorbed onto the linen from a solvent containing the nanoplates. Alternatively, threads soaked or printed with nanoparticles can be used in the manufacture of a linen or article. Upon subsequent exposure to moisture, saline, wound exudate, urine, or sweat the silver nanoplates undergo shape changes visually indicating the degradation of their plasmonic shape. The color of the nanoplates may indicate the amount of time that has expired since body moisture first contaminated the article (FIG. 22).

### Kits and Methods for Activation

In one embodiment an anti-microbial formulation comprising stabilized silver nanoplates is provided at a concentration of at least 1 mg/mL, at least 0.01, 0.1, 1, 10 mg/mL or from 0.01-0.1, 0.05-0.5, 0.1-1.0, 0.5-5.0 mg/mL, wherein the stabilized silver nanoplates are formulated such that when the concentration thereof is reduced 10 fold the encapsulation is susceptible to degradation. In some embodiments the stabilized nanoplates of the anti-micrbial formulation are coated in silica. In some embodiments, a kit is provided comprising the formulation and having one or more container housing a diluent. In some embodiments the diluent comprises water, an etchant, or a combination thereof. In one embodiment the etchants comprise one or more of salts (chlorine salt, halide salts, nitrate salts, sulfuric salts), bleach, sodium chloride, thiol or mercapto containing compounds, hydrogen sulfide, selenium, tellurium, oxygen, or hydrogen peroxide.

In one embodiment a kit is provided wherein the stabilized silver nanoparticles are present in a first container and the diluent is present in a second container, wherein the first container and the second container are operably linked such that the contents thereof are separated by a disruptable separation means comprising glass, plastic, or another suitable material. In one embodiment the kit comprises an applicator. In one embodiment the stabilized silver nanoplates are more stable than non-stabilized nanoplates at a temperature between 0 degrees C. to about 100 degrees C., e.g., about less than 5, 25, 30, 35, 40, 45, 50 or greater than 50 degrees C. for at least about 1 week, at least about 1 months, at least about 3 months, or greater than about 3 months.

In one embodiment, the composite does not release silver ions in the dry state and is only activated (e.g., to release silver ions) in the presence of moisture. The moisture can be from a high humidity environment, dipping or spraying the composite with a water based compound, or from the composite being in contact with a moist surface. Examples of moist surfaces include wounds such as burns, lacerations, ulcers, non-healing wounds, cuts, gun shot wounds, and injuries due to explosive fragmentation. Other types of surfaces that the composite can be applied to include clothing, foot wear, socks, wraps, compression bandages, porous surfaces (e.g. porous surfaces on furniture and equipment), medical devices, and other surfaces that need to be sterile.

In one embodiment, the metastable silver nanoparticles and the stability modulant have been optimized to release silver ions over an extended period of time. In some embodiments, the local concentration of silver ions in and around the composite when exposed to a moist environment for the first time is at least 5 ppb, 10 ppb, 20 ppb, 40 ppb 100 ppb, 300 ppb, 500 ppb, 1000 ppb, 2 ppm, 5 ppm, 10 ppm 40 ppm, or 100 ppm or more. In some embodiments the silver ion release rate is at least 20%, 30%, 50%, or 70% of the initial silver ion release rate value after 12 hours. In some embodiments, the silver on the composite is mostly retained after a wash step. In some embodiments, at least 30%, 50%, 80%, 90% or 95% of the initial silver is retained after a wash cycle of the composite.

### Combinations

In some embodiments an antimicrobial composition comprising stabilized silver nanoplates may further comprise an anti-fungal agent, an anti-microbial agent, an anti-viral agent, anti-inflammatory agent or a combination thereof

Antibacterial agents. Antibacterial agents include, without limitation, alcohol, aldehyde, anilide, diamidine, halogen-releasing agent, peroxygen, and/or phenols., bis-biguanide salts (e.g., chlorhexidine digluconate, chlorhexidine diacetate, chlorhexidine dihydrochloride, chlorhexidine diphosphanilate), rifampin, minocycline, silver compounds (silver chloride, silver oxide, silver sulfadiazine), triclosan, octenidin salts, octenidine dihydrochloride, quaternary ammonium compounds (e.g., benzalkonium chloride, tridodecyl methyl ammonium chloride, didecyl dimethyl ammonium chloride, chloroallyl hexaminium chloride, benzethonium chloride, methylbenzethonium chloride, cetyl trimethyl ammonium bromide, cetyl pyridinium chloride, dioctyldimethyl ammonium chloride), iron-sequestering glycoproteins (e.g., lactoferrin, ovotransferrin/conalbumin), cationic polypeptides (e.g., protamine, polylysine, lysozyme), surfactants (e.g., SDS, Tween-80, surfactin, Nonoxynol-9) and zinc pyrithione. Further preferred antimicrobial agents include broad-spectrum antibiotics (quinolones, fluoroquinolones, aminoglycosides and sulfonamides), and antiseptic agents (iodine, methenamine, nitrofurantoin, validixic acid). Octenidine dihydrochloride and bisbiguanide salts are preferred antimicrobial agents for use in the present invention, with chlorhexidine and its salts being particularly preferred. According to some aspects, chlorhexidine digluconate (CHG, chorohexadine gluconate) is used as the antimicrobial agent.

Antibacterial agents also include antibacterial drugs selected from the group comprising Aminoglycosides including Amikacin, Gentamicin, Kanamycin, Neomycin, Netilmicin, Tobramycin, Paromomycin, and Spectinomycin; Ansamycins including Geldanamycin, Herbimycin, Rifaximin, streptomycin; Carbacephem including and Loracarbef; Carbapenems including Ertapenem, Doripenem, ‘Imipenem’/Cilastatin, and Meropenem; Cephalosporins (First generation) including Cefadroxil, Cefazolin, ‘Cefalotin’ or Cefalothin, and Cefalexin; Cephalosporins (Second generation) including Cefaclor, Cefamandole, Cefoxitin, Cefprozil, and Cefuroxime; Cephalosporins (Third generation) including Cefixime, Cefdinir, Cefditoren, Cefoperazone, Cefotaxime, Cefpodoxime, Ceftazidime, Ceftibuten, Ceftizoxime, and Ceftriaxone; Cephalosporins (Fourth generation) including Cefepime; and Cephalosporins (Fifth generation) including Ceftaroline fossil, Ceftobiprole; Glycopeptides including Teicoplanin, Vancomycin, and Telavancin; Lincosamides including Clindamycin, and Lincomycin; Lipopeptide including Daptomycin; Macrolides including Azithromycin, Clarithromycin, Dirithromycin, Erythromycin, Roxithromycin, Troleandomycin, Telithromycin, and Spiramycin; Monobactams including Aztreonam; Nitrofurans including Furazolidone, Nitrofurantoin; Oxazolidonones including Linezolid, Posizolid, Radezolid, and Torezolid; Penicillins including Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; Penicillin combinations including Amoxicillin/clavulanate, Ampicillin/sulb actam, Piperacillin/tazobactam, and Ticarcillin/clavulanate; Polypeptides including Bacitracin, Colistin, and Polymyxin B; Quinolones including Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin; Sulfonamides including Mafenide, Sulfacetamide, Sulfadiazine, Silver sulfadiazine, Sulfadimethoxine, Sulfamethizole, Sulfamethoxazole, ‘Sulfanilimide’ (archaic), Sulfasalazine, Sulfisoxazole, ‘Trimethoprim’-Sulfamethoxazole (Co-trimoxazole) (TMP-SMX), and Sulfonamidochrysoidine (archaic); Tetracyclines including Demeclocycline, Doxycycline, Minocycline, Oxytetracycline, and Tetracycline; drugs against mycobacteria including Clofazimine, Dapsone, Capreomycin, Cycloserine, Ethambutol, Ethionamide, Isoniazid, Pyrazinamide, ‘Rifampicin’ (Rifampin in US), Rifabutin, Rifapentine, and Streptomycin; and others including Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim, and Fidaxomicin.

Antifungal agents. Antifungal agents are selected from the group comprising Polyene antifungals including Amphotericin B, Candicidin, Filipin, Hamycin, Natamycin, Nystatin, and Rimocidin; Imidazoles including Canesten (clotrimazole) anti fungal cream, Bifonazole, Butoconazole, Clotrimazole, Econazole, Fenticonazole, Isoconazole, Ketoconazole, Miconazole, Omoconazole, Oxiconazole, Sertaconazole, Sulconazole, and Tioconazole; Triazoles including Albaconazole, Fluconazole, Isavuconazole, Itraconazole, Posaconazole, Ravuconazole, Terconazole, and Voriconazole; Thiazoles including Abafungin; Allylamines including Amorolfin, Butenafine, Naftifine, and Terbinafine; Echinocandins including Anidulafungin, Caspofungin, and Micafungin; other agents including Benzoic acid, Ciclopirox, Flucytosine or 5-fluorocytosine, Griseofulvin, Haloprogin, Polygodial, Tolnaftate, Undecylenic acid, Crystal viol, Piroctone olamine, and Zinc pyrithione; and alternative agents and essential oils including Allicin, Citronella oil, Coconut oil, Iodine, Lemon myrtle, Neem seed oil, Olive leaf, Orange oil, Palmarosa oil, Patchouli, Selenium, Selenium sulfide, Tea tree oil, Zinc, Horopito (Pseudowintera colorata) leaf containing polygodia, Turnip, Chives, Radish, and Garlic.

Antiviral agents. Antivial agents include Abacavir, Aciclovir, Acyclovir, Adefovir, Amantadine, Amprenavir, Ampligen, Arbidol, Atazanavir, Atripla (fixed dose drug), Balavir, Boceprevirertet, Cidofovir, Combivir (fixed dose drug), Darunavir, Delavirdine, Didanosine, Docosanol, Edoxudine, Efavirenz, Emtricitabine, Enfuvirtide, Entecavir, Entry inhibitors, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Tea tree oil, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine

Anti-inflammatory agents. Anti-inflammatory agents include steroids, including glucocorticoids or corticosteroids; non-steroidal anti-inflammatory derivatives including aspirin, ibuprofen, naproxen, paracetamol, acetaminophen; immune selective anti-inflammatory derivatives (ImSAIDs) including submandibular gland peptide-T, phenylalanine-glutamine-glycine; and natural bio-active compounds including Plumbago.

### Carriers

Suitable carriers are provided for administration to mammalian subjects. Exemplary carrier forms are a liquid, gel, powder, solid, semi-solid, or emulsion form (e.g., as gels, pastes, ointments, creams, lotions, emulsions, suspensions or powders). The carrier can be formulated for application in drop, mist and aerosol forms. A liquid includes an aqueous or a non-aqueous liquid, and in some embodiments the carrier has a viscosity exceeding 100 centipoise (cP), such as 100, 200, 300, 400, 500, 600, 700, 800, 900, 1000 or above 1000 cP.

The silver nanoplates are formulated within the carrier. In preferred embodiments, the silver nanoplates are substantially uniformly distributed within the carrier, such that variability between regions of the carrier are minimized In preferred embodiments silver nanoparticles will remain uniformly distributed in a carrier over months or years. A liquid carrier with high viscosity (e.g. about more than the viscosity of water) can retain silver nanoparticles in a uniform distribution over months or years, whereas carriers with low viscosity (e.g about the viscosity of water) will have silver nanoparticles settle out over days or weeks. Settling rates are a function of the nanoparticle mass, encapsulation, surface functionality and carrier properties including viscosity or solvent. Additional materials may be included such as gelling agents such as carboxymethyl cellulose (CMC), polyvinyl alcohol (PVA), collagen, pectin, gelatin, agarose, chitin, chitosan, and alginate, wherein the gelling agent is present in an amount between about 0.01-20% w/v

Topical formulations are prepared to permit even spreading and absorption into the cutaneous surfaces. Examples include sprays, mists, aerosols, lotions, creams, solutions, gels, ointments, pastes, emulsions, and suspensions. The silver materials are mixed under sterile conditions with an acceptable carrier, and with any preservatives, buffers, or propellants, which may be required. Topical preparations can be prepared by combining the silver materials with conventional pharmaceutically acceptable diluents and carriers commonly used in topical dry, liquid, cream and aerosol formulations. Ointment and creams can, for example, be formulated with an aqueous or oily base with the addition of suitable thickening and/or gelling agents. Thickening agents include aluminum stearate, hydrogenated lanolin, and the like. In formulations where the silver materials are protected from contact with water, the materials can be formulated with an aqueous or oily base and will, in general, also include one or more of the following: stabilizing agents, emulsifying agents, dispersing agents, suspending agents, thickening agents, coloring agents, perfumes, and the like. Powders can be formed with the aid of any suitable powder base, e.g., talc, lactose starch and the like. Drops can be formulated with an aqueous base or non-aqueous base, and can also include one or more dispersing agents, suspending agents, solubilizing agents, and the like.

For topical administration, it is in some embodiments, beneficial to formulate the silver materials in carriers that prolong adherence of the silver nanoplates on the skin, or aid in deposition of the nanoplates in the skin. For example, the encapsulated silver particles are further coated with polymers that aid in their long-term adherence to skin, cloth or other surfaces. Such delivery aids deposited on the outer surface of silver materials include dextran, wherein the dextran has a molecular weight above 5 kD, preferably above 20 kD, a non-polysaccharide polymer, preferably an aminoplast polymer, or non-ionic polysaccharides selected from the group comprising: hydroxypropyl methyl cellulose, hydroxyethyl methyl cellulose, hydroxypropyl guar, hydroxyethyl ethyl cellulose or methyl cellulose. In some embodiments, the non-ionic polysaccharide has a molecular weight above 20 kD, more preferably above 100 kD. In some embodiments the coated silver nanoplates are provided in liquid soap compositions for washing skin that enhance their deposition onto the skin. For example, this can be achieved with soap-based liquid body and facial wash compositions using high solvent, low water compositions and incompletely neutralized fatty acids to help structure the compositions, all in combination with stabilized silver nanoplates and other agents that enhance their deposition. In one embodiment a liquid soap composition is provided comprising: (a) 10-50% by weight of a fatty acid blend of C₁₂-C₁₈ fatty acids in which the neutralization of fatty acid blend is between 70% and 90%; 10-40% by weight co-solvent; preferably less than about 18% by weight water; about 3 to 20% by weight emollient or occlusive oil; 0.0001 to 10% by wt. antimicrobial silver materials. Optionally, the material is modified by treatment with multivalent soap and/or a hydrophobic agent such as hydrophobically modified cationic, hydrophobically modified non-ionic polymer and mixtures thereof. In addition, makeup and other appearance-enhancing materials are added to the formulation. In some embodiments the silica encapsulant is modified. For example, hydrophobic modification of silica comprises bonding at least one C4 to C18 alkyl group, more preferably a C8H17 alkyl group to a silica atom. In some embodiments hydrophobically modified particle has a primary particle size from 1 nm to 100 nm, preferably from 5 nm to 70 nm. Such a composition may be topically applied as a method of treating various skin conditions.

Powder formulations can contain excipients such as starch, tragacanth, cellulose derivatives, polyethylene glycols, silicones, bentonites, silicic acid, and talc, or mixtures thereof In addition, powders and sprays also can contain excipients such as lactose, talc, silicic acid, aluminum hydroxide, calcium silicates and polyamide powder, or mixtures of these substances. Solutions of nanocrystalline noble metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such a chlorofluorohydrocarbons and volatile unsubstituted hydrocarbons, such as butane and propane. Materials to avoid in formulations of the present invention in amounts greater than 0.1% or greater than 0.01% w/v include chloride salts, aldehydes, ketones, long chain alcohols (with the possible exception of polyvinyl alcohols, preferably no greater than C₈-alcohols, and preferably no greater than C₆-alcohols), glycerol, and triethanolamine

Provided are unit doses containing the silver materials formulated as provided herein. Such a unit dose generally contains sufficient material for a single application, typically by use of a single use container, such as a glass or polymer (e.g., plastic) vial. Vials or other containers may include an applicator, such as a brush, pen or similar apparatus for dispensing and/or moving the formulated carriers on the skin or other intended surface. In some embodiments, the single unit dose contains a solvent solution, which may be mixed with the silver materials in a container provided therewith, or by other means known in the art. Some embodiments might use applicators using a puncturing means including U.S. Pat. Nos. 4,415,288; 4,498,796; 5,769,552; 6,488,665; and 7,201,525; and U.S. Pat. Pub. No. 2006/0039742. Each of the references is incorporated by reference, in its entirety, herein. In some embodiments applicators may use frangible ampoules such as U.S. Pat. Nos. 3,757,782; 5,288,159; 5,308,180; 5,435,660; 5,445,462; 5,658,084; 5,772,346; 5,791,801; 5,927,884; 6,371,675; and 6,916,133. Each of the references is incorporated by reference, in its entirety, herein. Alternatively, an applicator assembly may be used comprising: a head portion having a proximal end, a distal end, and an interior portion defining a fluid chamber; a container slidably coupled to the head portion; a breakable membrane sealing an end of the container; an application member attached to the distal end; and a hollow puncture mechanism, wherein the puncture mechanism is mounted in the interior portion of the head portion and an interior of the container is placed in fluid communication with the application member by way of a fluid conduit that is formed through the hollow puncture mechanism from the container to the fluid chamber when the container is axially translated toward the head portion and the puncture mechanism pierces the breakable membrane as described in Pat. Pub. No. 2012/069565. Each of the references is incorporated by reference, in its entirety, herein. In various embodiments, a carrier is formulated containing from 0.00005-10%, 0.00005-0.0005%, 0.0001-0.001%, 0.0005-0.005%, 0.001-0.01%, 0.005-0.05%, 0.01-0.1%, 0.05-0.5%, 0.1-1%, 0.5-5%, 1-10% or greater than 10% by weight of the stabilized silver nanoplates.”

### Articles of Manufacture

In some embodiments, the silver materials described herein are provided in concentrated solutions or dry powders, but in other embodiments the compositions are provided in a form already associated with a product, such as a product for use by a consumer. Such products include food preparation or storage products, e.g., bags, bins, containers, plates, utensils, cutlery, and the like. Other products include clothing or apparel products like hats, gloves, socks, etc. The silver materials can also be incorporated for anti-microbial purposes into an electronic product, such as a telephone, mobile phone, tablet, laptop computer, desktop computer and peripherals associated therewith, radios, televisions, and all sleeves, covers, and objects associated with electronic products. In some embodiments the silver materials are incorporated into water filtration products.

In some embodiments, the silver materials are embedded in a carrier to function as a deodorant, antiperspirant, soap, shampoo, anti-dandruff agent, anti-fungal cream, moisturizer, or cosmetic, or as a toothpaste, mouthwash or oral hygiene solution. In some embodiments the silver materials may be provided in a lubricant composition comprising an effective lubricating amount of neutralized C₈- C₂₂ fatty acid soap and a base sufficient to set the pH of the composition at from 8 to 11.

By way of non-limiting example, the silver materials can be provided in deodorant and/or antiperspirant compositions in the form of clear gelled sticks, opaque sticks comprising a blend of waxy material, or aerosol compositions for application to the human axillae, in particular, the underarms, to reduce malodor. In particular, the silver materials described in this invention provide antimicrobial activity over a sustained period in the gel and/or on the skin with low silver concentration, minimal haze or pigmentation, and/or uniform loading, particularly in a gel, in formulations that are superior to the results achieved with silver salts or soluble silver compound deposited on a synthetic oxidic support. In some embodiments the silver nanoplates and other compositions of the present invention provide pigment to the deodorant in the form of green, violet and/or blue highlights.

For example, a deodorant gel composition is provided which comprises:

(a) from 10 to 75% by weight water,

(b) a gelling agent comprising an alkali metal salt of a C₁₂ to C₂₄ fatty acid and, optionally, a co-gellant,

(c) stabilized encapsulated silver nanoplates, such that they are protected from degradation by the water present in the gel composition

(d) optionally, one or more emollients,

wherein the gel composition is in the form of a clear deodorant stick.

Desirably, the coated silver nanoplates are present in the subject compositions in an amount of from about 0.0001 to about 2% by weight. In one embodiment the silver particles are present in the subject compositions in an amount of from about 0.001 to about 1% by weight, such as 0.01 to 0.1%, or 0.1 to 1%. The amount of preference will depend, in part, on the desired strength of antimicrobial activity, as well as the degree of clarity desired in the gelled compositions, as in some compositions silver amounts in excess of 0.1% can impart significant pigment to the stick including blue or green pigmentation, such coloration is different at various nanoplate dimensions. Thus, in certain embodiments compositions containing 0.01% by weight or less of silver materials are desired. In one embodiment of interest, the gelled compositions of this invention contain from about 0.0001 to about 0.1%, more particularly from 0.001 to 0.1% by weight of such silver materials.

Oral formulations. In some embodiments the silver materials are formulated as an oral tablet, such as an oral extended-release tablet, or as an oral liquid suspension.

Ocular formulations. In other embodiments the silver materials are formulated for ocular applications, typically having isotonic and/or lubricative properties.

Household and cleaning supplies. The compositions provided herein can also be formulated as a surface cleaning agent, laundry detergent, adhesive, or paint.

Surface sealing. In one embodiment the coated silver nanoplates are added to a hard surface treatment composition comprising a base composition comprising: silver nanoparticles in an amount to provide anti-microbial properties to the surface, 20-75% by weight of a water soluble trivalent metal ion salt, wherein the trivalent metal ion salt is a salt of chloride, phosphate, nitrate, and/or sulphate; 20-75% by weight of a saturated C8-C24 fatty acid soap, and 5-20% by weight of a silicone oil; wherein the hard surface treatment composition has a pH of not more than 8 at a concentration of 1 to 50 g/L of the base composition in water. In some embodiments the base composition is a solid composition. In some embodiments the base composition is anhydrous. In other embodiments a liquid hard surface treatment composition is provided comprising a 1-50 g/L of the base composition and a solvent selected from water, an alcohol or mixtures thereof. In some embodiments the liquid composition is applied to a hard surface and left to dry. In other embodiments the composition renders a surface water repellant.

### Filtration Devices

In one embodiment, encapsulated silver nanoplates and other silver materials can be used for an antimicrobial membrane having ultrafiltration properties useful for purification of drinking water under gravity. Encapsulated silver nanoplates and other silver materials embedded in or coated on ultrafiltration membranes kill and immobilize microorganisms like cysts, protozoa, bacteria and virus which cause fouling that result in reduced flow of water through the membrane. By using the techniques described in this invention to modultate ion release from encapsulated silver nanoplates, e.g., silica-coated nanoplates, it is possible to produce an antimicrobial membrane that has ultrafiltration properties for water purification which requires less number of interventions and has higher lifetime, without producing any byproduct and yet is capable of delivering microbiologically safe water. Antimicrobial membranes having ultra filtration properties by simple in situ precipitation technique with simultaneous phase separation. For example, an antimicrobial membrane of the present invention comprises a fabric material integrally skinned with a composite comprising a thermoplastic polymer and encapsulated silver nanoplates, or other silver materials. Fabric is selected from cotton, polyester, polypropylene, polycotton, nylon or any other non-woven, woven or knitted fabric. In some embodiments the polymer is selected from polysulfones or polyvinylidenefluoride. In one embodiment the filter is a spirally wound layer of non-pleated fabric enveloped with spirally wound layer of pleated fabric, in a housing having an inlet and an outlet. In some embodiments A filter a block of activated carbon comprising activated carbon particles bound together with a polymeric binder that is positioned at the core enveloped by the spirally wound layer of non - pleated fabric and spirally wound layer of pleated fabric.

Generally, ultrafiltration membranes with encapsulated silver nanoplates and other silver materials can be produced by a) preparing a solution of encapsulated silver nanoplates and other silver materials in a suitable water miscible solvent having a water content less than 1%; b) adding a thermoplastic polymer to the solution of step (a); and c) coating the solution obtained after step (b) onto a fabric selected from cotton, polyester, polypropylene, polycotton, nylon or any other non-woven, woven or knitted fabric. In some embodiments a suitable solvent is selected from N-methylpyrrolidone, dimethylformamide, dimethyl sulphoxide, dimethylacetamide and mixtures thereof.

Water purification kits. In one embodiment encapsulated silver nanoplates and other silver materials are introduced into water to kill unwanted microbes. Prior to introduction into water encapsulated silver nanoplates and other silver materials are in a composition that stabilizes nanoplate or other nano shape (e.g. dried/anhydrous on a table, as a film, in concentrated form with stabilizing coatings and buffer) upon dilution into water the encapsulated silver nanoplates and other silver materials degrade to release free ion at a concentration from 0.001 to 500 ppm. In one embodiment encapsulated silver nanoplates and other silver materials may be provided in a kit with instructions for use. In one embodiment encapsulated silver nanoplates may be provided with organic ligands (combined in solution or in a kit) which are able to form a water-soluble co-ordination complex with the silver ions that are released. Final organic ligand concentration in water may range 0.005 to 3000 ppm and could include a amphoteric or zwitterionic surfactant, a polyether, or a polycarboxylate or oligomer or polymer of one or more olefinically unsaturated monomers, and which contains an average of at least 1 carboxylate group per monomer residue.

Biopolymer stabilization. In some embodiments encapsulated silver nanoplates and other silver materials are in liquid compositions of biopolymers to reduce their susceptibility to microbial attack. Biopolymers are very abundant naturally occurring, or easily derived from naturally occurring, chemicals and their use in consumer products, such as liquid detergent formulations, is attractive from both environmental and cost grounds. Accordingly, they have been proposed for several applications in such compositions, including thickening or other rheological duties. In some embodiments biopolymers include: Microcrystalline cellulose, acetyl cellulose, and chitin. Encapsulated silver nanoplates and other silver materials can be synthesized in liquid compositions of biopolymers with biopolymers acting as reducing and stabilizing agents or encapsulated silver nanoplates and other silver materials can be combined with liquid compositions of biopolymers after synthesis.

### Methods of Treatment

The antimicrobial and anti-inflammatory compositions of the present invention are useful for treating several diseases and disorder. A method of treating a skin disease, disorder, or condition is provided wherein an area of the skin showing symptoms of the skin disease, disorder, or condition is contacted with a composition comprised of stabilized silver nanoplates. In one embodiment the composition contains from about 0.00005 weight percent to about 20 weight percent (e.g., 0.001-20, 1-5, 5-15) weight percent of stabilized silver nanoplates. In some embodiments the composition is in the form of a gel, a cream, a paste, an ointment, a lotion, an emulsion, a suspension or a liquid. In some embodiments the composition further comprises an anti-inflammatory, anti-viral, anti-bacterial, or anti-fungal agent. In some embodiments the skin condition is a form of eczema selected from the group consisting of atopic eczema, acrodermatitis eczema, contact allergic dermatitis, dyshydrotic eczema, lichen simplex chronicus, nummular eczema, and statis eczema. In some embodiments the skin condition is a form of an instect bite, an insect sting, an sunburn, a mycosis fungiodes, a pyoderma gangrenosum, rosacea, acne. In some embodiments, the composition is formulated as a topical solution, spray, mist, or drops containing 0.00005-10%, 0.00005-0.0005%, 0.0001-0.001%, 0.0005-0.005%, 0.001-0.01%, 0.005-0.05%, 0.01-0.1%, 0.05-0.5%, 0.1-1%, 0.5-5%, 1-10% or greater than 10% by weight of the stabilized silver nanoplates.

In some embodiments the composition is the form of a wound dressing. In some embodiments the composition comprises a hydrated dressing is selected from the group consisting of a hydrocolloid, hydrogel, polyethylene, polyurethane, polyvinylidine, siloxane and silicone dressing. The hydrocolloid dressing may contain a hydrocolloid selected from the group consisting of alginates, starch, glycogen, gelatin, pectin, chitosan, chitin, cellulose and derivatives thereof, gum Arabic, locust bean gum, karaya gum, gum tragacanth, ghatti gum, agar-agar, carrageenans, carob gum, guar gum, xanthan gum, and glyceryl polymethacrylate. In one embodiment the hydrocolloid is one or more of carboxymethyl cellulose, alginates, pectin and glyceryl polymethacrylate.

The antimicrobial and anti-inflammatory compositions of the present invention are useful for reducing inflammation or infection of a mucosal membrane, comprising: contacting an inflamed or infected problem area of the mucosal membrane with a therapeutically effective amount of a composition comprising stabilized silver nanoplates. Mucosal membranes include one or more of the oral cavity, the nasal, bronchial, pulmonary, trachea and pharynx airways, the otic and ophthalmic surfaces, the urogenital system, the reproductive system, and the gastrointestinal tract including the prostate, the colon or rectal surfaces.

Consumer Signaling.

An important aspect of the present invention is the ability of the compositions described to signal to consumers, practitioners, doctors, nurses, caregivers, and other professionals about the status or expiration of a device or product. The unique visible detection properties of stabilized encapsulated silver nanoplates that undergo shape changes while disposed in various formulation or surfaces provide real-time information about product characteristics that has important benefits for the user. For example, a dressing can signal to a patient when wound exudate is striking through causing the patient to be more compliant in frequent dressing changes. Alternatively, a continuous use indicator on an IV set can signal to a nurse that an IV set has exceeded a minimum usage and should be changed before it reaches its maximum. In some embodiments the consumer signaling is associated with the status or expiration of an antimicrobial effect. In other embodiments the consumer signaling demonstrates that time in which the product has been in use or the time that has expired since a product was activated, independent of antimicrobial effects. This novel effect may be applied to a host of articles and devices that have been described or may be appreciated in the future to make them more consumer friendly or to increase safe, compliant use of products.

### Cytotoxic and Cytostatic Formulations and Articles.

Preferably stabilized silver nanoplates are included in or on the articles in amounts that are cytotoxic, or cytostatic, meaning the silver materials are present in amounts adequate to kill or restrict the growth of one or more of the following microbes: coagulase-negative Staphylococci, Enterococci, fungi, Candida albicans, Staphylococcus aureus, Enterobacter species, Enterococcus faecalis, Staphylococcus epidermidis, Streptococcus viridans, Escherichia coli, Klebsiella pneumoniae, Proteus mirabilis, Pseudomonas aeruginosa, Acinetobacter baumannii, Burkholderia cepacia, Varicella, Clostridium difficile, Clostridium sordellii, Hepatitis A, Hepatitis B, Hepatitis C, HIV/AIDS, methicillin-resistant Staphylococcus aureus (MRSA), mumps, norovirus, parvovirus, poliovirus, rubella, SARS, S. pneumoniae (including drug resistant forms), vancomycin-intermediate Staphylococcus aureus (VISA), vancomycin-resistant Staphylococcus aureus (VRSA), and vancomycin- resistant Enterococci (VRE). It is considered to be within the ability of one skilled in the art to determine such amounts. Preferably stabilized silver nanoplates are included in or on the articles in amounts that are adequate to kill or restrict the growth of bacterial spores.

### Methods of Fabrication

Shaped silver nanoparticles are fabricated using methods known in the literature. For example, silver nanoplates can be fabricated using photoconversion (Jin et al. 2001; Jin et al. 2003), pH controlled photoconversion (Xue 2007), thermal growth (Hao et al. 2004; Hao 2002; He 2008; Metraux 2005), templated growth (Hao et al. 2004; Hao 2002), seed mediated growth (Aherne 2008; Chen; Carroll 2003; Chen; Carroll 2002, 2004; Chen et al. 2002; He 2008; Le Guevel 2009; Xiong et al. 2007), or alternative methods. See, e.g.:

Aherne, D. L., D. M.; Gara, M.; Kelly, J. M., 2008: Optical Properties and Growth Aspects of Silver Nanoprisms Produced by Highly Reproducible and Rapid Synthesis at Room Temperature. Advanced Materials, 18, 2005-2016.

Chen, S., and D. L. Carroll, 2003: Controlling 2-dimensional growth of silver nanoplates. Self-Assembled Nanostructured Materials Symposium (Mater. Res. Soc. Symposium Proceedings Vol.775), 343-348|xiii+394.

Chen, S. H., and D. L. Carroll, 2002: Synthesis and characterization of truncated triangular silver nanoplates. Nano Letters, 2, 1003-1007.

Chen, S. H., and D. L. Carroll, 2004: Silver nanoplates: Size control in two dimensions and formation mechanisms. Journal of Physical Chemistry B, 108, 5500-5506.

Chen, S. H., Z. Y. Fan, and D. L. Carroll, 2002: Silver nanodisks: Synthesis, characterization, and self-assembly. Journal of Physical Chemistry B, 106, 10777-10781.

Hao, E., G. C. Schatz, and J. T. Hupp, 2004: Synthesis and optical properties of anisotropic metal nanoparticles. Journal of Fluorescence, 14, 331-341.

Hao, E. K., K. L.; Hupp, J. T.; Schatz, G. C., 2002: Synthesis of Silver Nanodisks using Polystyrene Mesospheres as Templates. J Am Chem Soc, 124, 15182-15183.

He, X. Z., X.; Chen, Y.; Feng, J., 2008: The evidence for synthesis of truncated silver nanoplates in the presence of CTAB. Materials Characterization, 59, 380-384.

Jin, R., Y. Cao, C. A. Mirkin, K. L. Kelly, G. C. Schatz, and J. G. Zheng, 2001: Photoinduced Conversion of Silver Nanospheres to Nanoprisms. Science, 294, 1901-1903.

Jin, R., Y. C. Cao, E. Hao, G. S. Metraux, G. C. Schatz, and C. A. Mirkin, 2003: Controlling anisotropic nanoparticle growth through plasmon excitation. Nature, 425, 487.

Le Guevel, X. W., F. Y.; Stranik, O.; Nooney, R.; Gubala, V.; McDonagh, C.; MacCraith, B. D., 2009: Synthesis, Stabilization, and Functionalization of Silver Nanoplates for Biosensor Applications. J Phys Chem C, 113, 16380-16386.

Metraux, G. S. M., C. A; , 2005: Rapid Thermal Synthesis of Silver Nanoprisms with Chemically Tailorable Thickness. Advanced Materials, 17, 412-415.

Xiong, Y. J., A. R. Siekkinen, J. G. Wang, Y. D. Yin, M. J. Kim, and Y. N. Xia, 2007: Synthesis of silver nanoplates at high yields by slowing down the polyol reduction of silver nitrate with polyacrylamide. Journal of Materials Chemistry, 17, 2600-2602.

Xue, C. M., C. A., 2007: pH-Switchable Silver Nanoprism Growth Pathways. Angew Chem Int Ed, 46, 2036-2038.

Each of the references listed above is incorporated by reference in its entirety, herein.

Alternative methods include methods in which the silver nanoparticles are formed from a solution comprising a shape stabilizing agent or agents and a silver source, and in which chemical agents, biological agents, electromagnetic radiation, or heat are used to reduce the silver source. Synthesis methods for other shapes and sizes of silver nanoparticles are reported in the scientific literature.

Use of Materials with Compositions and Products

In some embodiments, the silver materials of the present invention can be incorporated into compositions, products, substrates, surfaces, etc. that are described in, e.g., the following publications: WO2013090440, WO2013142692, WO2013090615, CA2765393, US2012/037163, WO2012161954, EP2011/063939, EP2011/063939, WO2013064365, WO2013026657, WO2013026656, WO2013017393, WO2012156170, WO2011128248, EP2230321, US20100158841, WO2010057968, WO2010046354, CA2554112, CA2601346, WO2005075547, WO2005073296, WO1999061567, WO1996001231, EP0678548, CA2075238, CA2003972, EP0373688, EP0049830, CA2085956, EP0551674, CA2085956, WO1995002392. Each of the references listed above is incorporated by reference in its entirety, herein.

Other embodiments of the invention will be apparent to those skilled in the art from consideration of the specification and practice of the invention disclosed herein. It is intended that the specification and examples be considered as disclosing certain embodiments of the invention only, with a true scope and spirit of the invention being indicated by the following claims.

The subject matter described herein may be embodied in other specific forms without departing from the spirit or essential characteristics thereof The foregoing embodiments are therefore to be considered in all respects illustrative rather than limiting. While embodiments are susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited.

The methods disclosed herein include certain actions taken by a practitioner; however, they can also include any third-party instruction of those actions, either expressly or by implication. For example, actions such as “application to a target region of skin tissue” include “instructing the application to a target region of skin tissue.”

The ranges disclosed herein also encompass any and all overlap, sub-ranges, and combinations thereof Language such as “up to,” “at least,” “greater than,” “less than,” “between,” and the like includes the number recited. Numbers preceded by a term such as “about” or “approximately” or “substantially” include the recited numbers. For example, “about 3 mm” includes “3 mm.” The terms “approximately”, “about” and/or “substantially” as used herein represent an amount or characteristic close to the stated amount or characteristic that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount or characteristic.

### EXAMPLES

The description of specific examples below are intended for purposes of illustration only and are not intended to limit the scope of the invention disclosed herein.

### Example 1

### Silver Nanoplates

Silver nanoplates were synthesized using silver seeds prepared through the reduction of silver nitrate with sodium borohydride in the presence of sodium citrate tribasic and poly sodium styrene sulfonate under aqueous conditions. Silver seed preparation: 21.3 mL of an aqueous 2.5 mM sodium citrate tribasic solution was allowed to mix under magnetic stirring. 1 mL of a 2 g/L poly styrene sodium sulfonate (PSSS) solution was then prepared in a separate beaker. 21.3 mL of a 0.5 mM silver nitrate solution was then prepared by dissolving the salt in water. Once the above solutions have been prepared, 1.33 mL of a 0.5 mM sodium borohydride solution should be prepared using cold water. The borohydride and PSSS solutions were then added to the beaker containing the citrate and allowed to mix. The silver nitrate solution was then pumped into the citrate solution using a peristaltic pump at a rate of 100 mL/min. This seed solution was then allowed to stir overnight at room temperature. Silver nanoplate preparation: Silver nanoplates were prepared by mixing 1530 mL Milli-Q water with 35 mL of a 10 mM ascorbic acid solution. Once the solution sufficiently mixed, the silver seed (made 24 h prior) was added to the beaker. 353 mL of a 2 mM silver nitrate solution was then pumped into the beaker at a rate of 100 mL/min. Following the completion of the silver nitrate, the solution was allowed to mix at room temperature for at least two hours to allow the reaction to go to completion.

### Example 2

### Silica Encapsulation of Silver Nanoplates (e.g., Shelling)

A silica shell was grown on the surface of 800 nm resonant (˜75 nm diameter polyvinylpyrolidone (PVP) capped silver nanoplates. 600 mL of a solution of 800 nm resonant PVP40T capped silver nanoplates at a concentration of 1 mg/mL was added to 3.5 L of reagent grade ethanol and 270 mL Milli-Q water under constant stirring. 4.3 mL of dilute aminopropyl triethoxysilane (215 uL APTES in 4.085 mL isopropanol) was then added to the solution, followed immediately by the addition of 44 mL of 30% ammonium hydroxide. After 15 minutes of incubation, 31 mL of dilute tetraethylorthosilicate (1.55 mL TEOS in 29.45 mL, isopropanol) was added to the solution. The solution was then left to stir overnight. The nanoplates were then centrifuged on an Ultra centrifuge at 17000 ref for 15 min and reconstituted in milli-Q water each time and repeated twice. The shell thickness was controlled by the amount of TEOS added.

### Example 3

### Binding to a Substrate

10 mL of silver nanoplates prepared at a concentration of 1 mg/mL were incubated with a 5 g coupon from a commercially available chamois (Detailer's Choice). The fluid was completely absorbed by the chamois and allowed to air dry to produce a darkly colored substrate.

### Example 4

### Addition of a Stability Modifier

10 mL of silver nanoplates prepared at a concentration of 1 mg/mL were incubated with a 5 g coupon from a commercially available chamois (Detailer's Choice). The fluid was completely absorbed by the chamois and allowed to air dry to produce a darkly colored substrate. The dried coupon was incubated with 3 mL of a 1 M solution of NaCl and heat dried to produce a substrate with a stability modifier dried into the sample.

### Example 5

### Silver Ion Release Rates

The silver ion concentration of 1 mg/mL 10 nm silver nanoparticles was measured to be 3 ppb within 12 hours of synthesis and increased to 22 ppb after 4 days. The silver ion concentration of silver nanoplates in a sodium borate buffer was 9 ppb after 2 days. The silver ion concentration of silver nanoplates in a water solution was 1160 ppb after 1 day.

### Example 6

### Antimicrobial Filter Membrane with Silica Encapsulated Silver Nanoplates

Silica encapsulated nanoplates were synthesized according to the methods described in Example 1 and 2 and resuspended in 100 mL of N-methylpyrrolidone (NMP) (Merck, 99.99%) at a concentration of 1% silver using ultracentrifugation. To this solution 15 g of polysulfone [PSf, Aldrich, Number average molecular weight=26,000; Tg (glass transition temperature)=195° C.] was added in parts with continuous stirring by using an overhead stirrer while keeping the solution temperature at about 70° C. until the PSf was fully disperse. A pleated polyester fabric was kept horizontally on the table top and two ends were clipped properly. One of the surfaces of the fabric was coated with polymer solution [PSf:Ag:NMP=15:1:100] uniformly by brush-paint method. Similarly, the spiral fabric was also coated. Then the composite fabric was dried inside air-oven at 50-60° C.

A small aliquot of the polymer solution was deposited in a 96-well plate and dried inside an air-oven at 50-60° C. The polymer solution was analyzed by a spectrophotomer, which showed no appreciable shift in the peak resonance wavelength, confirming that the silver nanoplates had been incorporated into the polymer in a stabilized form, wherein its high curvature shape was retained.

### Example 7

### Filter with an Antimicrobial Membrane with Encapsulated Silver Nanoplates

Both ends of a pleated antimicrobial membrane having 45 pleats (each side of pleat is 10 mm) width of 5.7 cm and thickness of about 2 mm were sealed with polyethylene based Hot Melt Adhesive (HMA), to form a tubular pleated membrane. Same process was done for spiral antimicrobial membrane having width of 5.7 cm and thickness of about 2 mm. Then spiral fabric was wound over a perforated plastic tube having diameter of 3 cm and length 5.7 cm such that a total 6 number of spiral layers are incorporated resulting a length of 106 cm and then the open end of the fabric was sealed with polyethylene based HMA. This assembly was then covered with pleated antimicrobial membrane made above, such that the pleated and the spirally wound membranes are concentric. Then on a plastic plate of diameter 10 cm, polyethylene based HMA was poured starting from edge and up to the serration mark of about 2 mm thick in spiral manner and the composite assembly was fixed over it and allowed the HMA to cool for about 2-3 minutes under 2 kg pressure. Similarly the other end of the assembly was fixed with another similar piece of plastic. After cooling the filter was ready.

Water easily flowed through the silver with no detectable silica coated nanoplates coming off into the filtrate when 1 mL of water was passed through the filter and visualized via spectrophotometry. The silica coated nanoplates embedded in the filter system were detectable by eye based on their dark blue/indigo hue.

### Example 8

### Gelled Deodorant Sick with Silica Stabilized Silver Nanoplates

Gelled sticks with silver nanoplates were prepared by blending water, dipropylene glycol and propylene glycol components, heating the resulting blends to 85° C., adding the poloxamine to the heated blend and mixing until clear, adding the sodium stearate to the heated blends and again mixing until clear, cooling the blends to 75° C. and adding the 2-amino-2-methylpropan-1-ol with agitation, cooling the blends to 71-73° C., and mixing in the remaining ingredients. Non-stabilized silver nanoplates and silica stabilized silver nanoplates were prepared according to the methods in example 1 and 2 respectively and added into separate blends. A silver salt was added to a separate blend. The blends were then allowed to cool to room temperature and gel. Compositions of the three blends are shown in Table A.

TABLE A

| | Wt. % | | |
|---|---|---|---|
| | SILICA | | |
| | STABILIZED | NON- | |
| | SILVER | STABILIZED | |
| Component | NANOPLATES | NANOPLATES | SALT |
| Water | QS | QS | QS |
| Propylene Glycol | 22.5% | 22.5% | 22.5% |
| Dipropylene Glycol | 40.0% | 40.0% | 40.0% |
| Sodium Stearate | 5.5% | 5.5% | 5.5% |
| Tetronic ® 1307 | 3.0% | 3.0% | 3.0% |
| Poloxamine | | | |
| Disodium EDTA | 0.1% | 3.0% | 3.0% |
| 2-amino-2- | 0.4% | 0.4% | 0.4% |
| methylpropan-1-ol | | | |
| BHT | 0.05% | 0.05% | 0.05% |
| Fragrance | 1.5% | 1.5% | 1.5% |
| Colorant | 0.005% | 0.005% | 0.005% |
| Silica stabilized | As 0.0005% | — | — |
| silver nanoplates | silver | | |
| Non-stabilized | — | As 0.0005% | — |
| silver nanoplates | | silver | |
| Silver Chloride | | | As 0.0005% |
| powder | | | silver salt |

Salt blends exhibited settling of the silver chloride, whereas, settling of the silica stabilized silver nanoplates particles and non-stabilized silver nanoplates was not observed. A 100 microliter aliquot of from non-stabilized and silica stabilized silver blend solutions was added to a 96 well plate and allowed to cool to room temperature and gel. The polymer solution was analyzed by a spectrophotomer which confirmed no appreciable shift in the peak resonance wavelength for silica stabilized silver nanoplates, but a significant shift in the peak plasmon resonance for the gel containing non-stabilized silver nanoplates. The shape degradation of the blend with non-stabilized silver nanoplates was visibly detectable as silica stabilized silver nanoplate blends have a faint blue hue while non-stabilized silver nanoplate blends shift from a faint blue to a yellow/orange hue.

This example confirms that stabilized silver nanoplates with high curvature can be incorporated into a gelled deodorant stick. Shape changes of silver nanoplates observed in non-stabilized blends confirm that the addition of stability modulants is a critical step to achieving the compositions of the present invention.

### Example 9

### Gelled Deodorant Sick with PVP and Borate Stabilized Silver Nanoplates

Gelled sticks with silver nanoplates were prepared by blending water, dipropylene glycol and propylene glycol components, heating the resulting blends to 85° C., adding the poloxamine to the heated blend and mixing until clear, adding the sodium stearate to the heated blends and again mixing until clear, cooling the blends to 75° C. and adding the 2-amino-2-methylpropan-1-ol with agitation, cooling the blends to 71-73° C., and then mixing in the remaining ingredients. PVP and borate stabilized silver nanoplates were prepared according to the methods in example 1 with the addition of PVP and borate into the mixture after synthesis. In one blend was added borate prior to adding silver nanoplates, in another blend borate was not added. A silver salt was added to a separate blend with no borate. The blends were then allowed to cool to room temperature and gel. Compositions of the three blends are shown in Table B.

TABLE B

| | Wt. % | | |
|---|---|---|---|
| | STABILIZED | NON- | |
| | SILVER | STABILIZED | |
| Component | NANOPLATES | NANOPLATES | SALT |
| Water | QS | QS | QS |
| Propylene Glycol | 22.5% | 22.5% | 22.5% |
| Dipropylene Glycol | 40.0% | 40.0% | 40.0% |
| Sodium Stearate | 5.5% | 5.5% | 5.5% |
| Tetronic ® 1307 | 3.0% | 3.0% | 3.0% |
| Poloxamine | | | |
| Disodium EDTA | 0.1% | 3.0% | 3.0% |
| 2-amino-2- | 0.4% | 0.4% | 0.4% |
| methylpropan-1-ol | | | |
| BHT | 0.05% | 0.05% | 0.05% |
| Fragrance | 1.5% | 1.5% | 1.5% |
| Colorant | 0.005% | 0.005% | 0.005% |
| PVP stabilized | As 0.0005% | — | — |
| silver nanoplates | silver | | |
| Borate | 0.05% | — | — |
| Silver Chloride | | | As 0.0005% |
| powder | | | silver salt |

Salt blends exhibited settling of the silver chloride, whereas, settling of the silica stabilized silver nanoplates particles and non-stabilized silver nanoplates was not observed. A 100 microliter aliquot from non-stabilized and silica stabilized silver blend solutions was added to a 96 well plate and allowed to cool to room temperature and gel. The polymer solution was analyzed by a spectrophotomer which confirmed no appreciable shift in the peak resonance wavelength for PVP stabilized silver nanoplates in a deodorant stick containing borate, but a significant shift in the peak plasmon resonance for the gel containing PVP silver nanoplates in a deodorant carrier in which no borate was added. The shape degradation of the blend with non-stabilized silver nanoplates was visibly detectable as stabilized silver nanoplate blends have a faint blue hue while non-stabilized silver nanoplate blends shift from a faint blue to a yellow/orange hue.

This example confirms that stabilized silver nanoplates with high curvature can be incorporated into a gelled deodorant stick. Shape changes of silver nanoplates observed in non-stabilized blends confirm that the addition of stability modulants is a critical step to achieving the compositions enabled by the present invention.

### Example 10

### Pressure Sensitive Adhesive Containing Silica Coated Silver Nanoplates

Silica coated silver nanoplates synthesized according the methods in example 1 and 2 and resuspended in alcohol. The mixture ultracentrifuged to a form a small pellet of silver nanoplates in an ultracentifuge tube and the supernatant was substantially removed. The pellet was then resuspended in an adhesive matrix by mixing and readily forms a suspension in the adhesive matrix. The formulation details are as follows:

Ag—SiO₂ 0.1 grams

Duro-Tak 87-900A adhesive 97.0 grams (National Starch and Chemical, Bridgewater, N.J.)

The adhesive solution was analyzed by a spectrophotomer and confirmed no appreciable shift in the peak resonance wavelength of the silver nanoplates in the adhesive matrix indicating that silver nanoplates were incorporated in a stabilized form.

### Example 11

### Medical Suture Coated with Encapsulated Silver Nanoplates

A medical suture material size 2/0, polyester braid was coated by dipping the suture into a 10 mg/mL solution of silica coated silver nanoplates prepared according to the methods of example 1 and 2 with an additional concentrating step in water. The braid was removed from the solution and dried inside an air-oven at 50-60° C. and then placed in a sealed container with desiccant.

Two sutures were analyzed, one after 1 day and the other after 3 months in a sealed container. The sutures were placed in 1 mL of water in a microcentrifuge tube and allowed to incubate for 6 hours. Afterwards a small aliquot was taken from the supernatant of the solution and analyzed by spectrophotometry. There was no appreciable shift in the peak resonance wavelength of the silver nanoplates detected in the supernatant of the suture incubated for 1 day and 3 months. The encapsulated silver nanoplates remained stable in a moisture free environement for several months. Afterwards the supernatant solutions were followed for several days, wherein detectable shifts of the peak plasmonic wavelength were observed according to the sustained release profile anticipated from a silica encapsulated silver nanoplate in water.

### Example 12

### Catheter Coated with Encapsulated Silver Nanoplates

A teflon coated latex Foley catheter was coated by dipping the catheter into a 10 mg/mL solution of silica coated silver nanoplates prepared according to the methods of example 1 and 2 with an additional concentrating step in water. The catheter was removed from the solution and dried inside an air-oven at 50-60° C. and then placed in a sealed container with desiccant.

Two catheters were analyzed, one after 1 day and the other after 3 months in a sealed container. The catheters were placed in 50 mLs of water in a glass beaker and allowed to incubate for 6 hours. Afterwards a small aliquot was taken from the supernatant of the solution and analyzed by spectrophotometry. There was no appreciable shift in the peak resonance wavelength of the silver nanoplates detected in the supernatant of the catheter incubated for 1 day and 3 months. The encapsulated silver nanoplates remained stable in a moisture free environment on the catheter for several months. Afterwards the supernatant solutions were followed for several days, wherein detectable shifts of the peak plasmonic wavelength were observed according to the sustained release profile anticipated from a silica encapsulated silver nanoplate in water.

### Example 13

### Wound Dressing Material with Encapsulated Silver Nanoplates

This example is included to demonstrate a multilayer burn wound dressing in accordance with the present invention. High density polyethylene mesh dressing material CONFORMANT 2™ dressing was soaked in a 10 mg/mL solution of silica coated silver nanoplates prepared according to the methods of example 1 and 2 with an additional concentrating step in water. The dressing material was removed from the solution and dried inside an air-oven at 50-60° C. and then placed above and below an absorbent core material formed from needle punched rayon/polyester (SONTARATM 8411). The three layers were laminated together by ultrasonic welding to produce welds between all three layers spaced at about 2.5 cm intervals across the dressing. The laminated dressing was placed in a sealed container with a desiccant.

After three months the dressing was removed from the sealed container and placed in 50 mLs of water in a glass beaker and allowed to incubate for 6 hours. Afterwards a small aliquot was taken from the supernatant of the solution and analyzed by spectrophotometry. There was no appreciable shift in the peak resonance wavelength of the silver nanoplates detected in the supernatant of the dressing incubated 3 months. Afterwards the supernatant solution was followed for several days, wherein detectable shifts of the peak plasmonic wavelength were observed according to the sustained release profile anticipated from a silica encapsulated silver nanoplate in water.

### Example 14

### Gels with Stabilized Silver Nanoplates for Wound Treatment

A 20 mg/mL solution of silica coated silver nanoplates in water was prepared according the methods in example 1 and 2 with an additional concentration step. A gel-based carrier solution comprising 37% water, 40% propylene glycol, 2% SDS, 0.5% PE 9010 preservative and Aristoflex AVC polymer was mixed with the silver nanoplate solution at 1:1 ratio. The final viscosity of the solution was about 1000 cP. The material was loaded into a lml syringe for topical use (Baxter, Baxa) and stored at 4 deg C for a year. Fifty microliter aliquots were removed from the solution immediately after formulation and every 3 months for up to 12 months to be analyzed spectrophotometrically. There was no appreciable shift in the peak resonance wavelength of the silver nanoplates in the carrier gel solution for at least 12 months. The particles remained dispersed in the solution such that the concentration of silver nanoplates in each aliquot remained the same. The solution color, dark indigo remained stable for at least one year.

The silver nanoplate gel was administered to a 33 year old male patient with skin on the left thigh that had been treated by a fractionated laser (Fraxel 1.5 mm deep posts at 15% coverage). A vibraderm massage device was used to massage the gel on the treated skin for 5 min to embed silver nanoplates into the fractionated skin. Afterwards, stabilized silver nanoplates appeared in each of the ablated wells on the skin from the fractionated laser with a dark blue punctate pattern. The particles were verified as embedded within the wells as they could not be removed with soap and water washes or alcohol wipes. The bright blue pattern was sustained for about 2 days, but after day 1 the hue began to shift from blue to yellow and silver, representing a shape change of the particle and a sustained release of silver ions over time. By day 3 there was no more hue present in the skin, confirming that the silver particles had fully dissolved. There were no infections or adverse inflammatory responses and the skin healed completely.

### Example 15

### Bioabsorbable Sutures with Encapsulated Silver Nanoplates

A bioabsorbable medical suture, DEXON™ II BI-COLOR (Braided polyglycolic acid with polycaprolate coating) was coated by dipping the suture into a 10 mg/mL solution of silica coated silver nanoplates prepared according to the methods of example 1 and 2 with an additional concentrating step in water. The suture was removed from the solution and dried inside an air-oven at 50-60° C. and then placed in a sealed container with desiccant.

After 3 months in a sealed container the sutures were placed in 3 mLs of saline in a microcentrifuge tube and allowed to dissolve. Small aliquots were taken from the supernatant periodically and analyzed by spectrophotometry. There was no appreciable shift in the peak resonance wavelength of the silver nanoplates detected in the supernatant immediately after the suture was placed in the saline solution. Afterwards the supernatant solutions were followed for several days, wherein detectable shifts of the peak plasmonic wavelength were observed according to the sustained release profile anticipated from a silica encapsulated silver nanoplate in saline. Furthermore, the absorbable polymer was also seen to degrade in solution over the course of 2 weeks.

### Example 16

### Antimicrobial Solution with Colorimetric Signaling

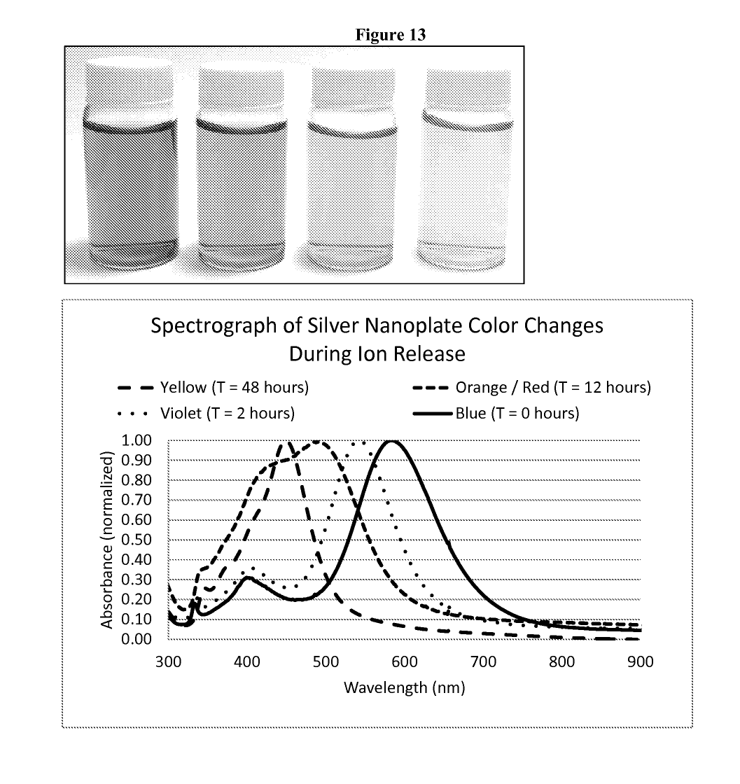

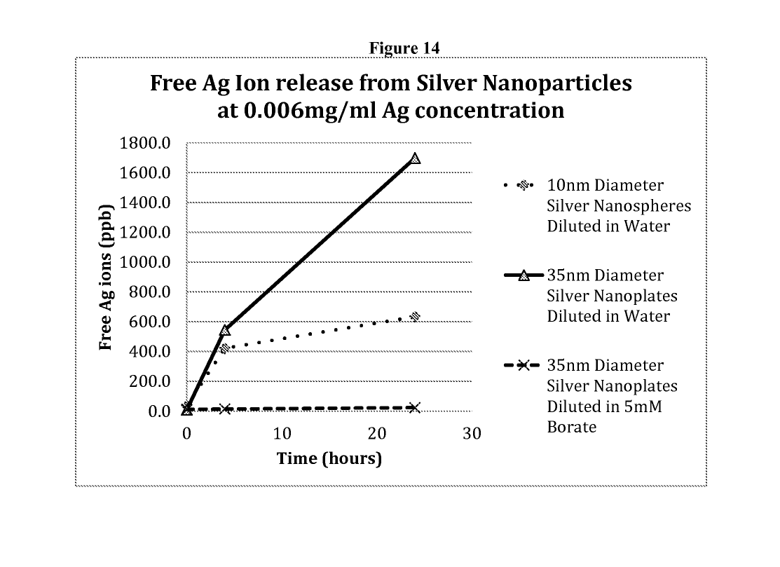

A solution of PVP coated silver nanoplates (˜35 nm diameter and ˜10 nm in thickiness) in 5 mM borate and water at a concentration of 1.3 mg/mL silver is diluted into water to a concentration of 0.006 mg/mL silver. The initial solution color is blue, but changes from blue to violet within 2 hours, to red/orange within 12 hours, and settles at a final color of yellow within 48 hours. The color shift is due to changes in the nanoplate dimension, namely a reduction in average diameter of the nanoplates and a reduction in their aspect ratio. The solution is maintained in a transparent vial to monitor color changes due to ion release from the silver nanoplates. Results are shown in FIG. 13

A corresponding ion release profile is measured for the silver nanoplates that undergo color changes. Ion release rates of PVP coated silver nanoplates (35 nm diameter, 10 nm thickness) diluted into water to a concentration of 0.006 mg/mL are compared to 1) PVP coated silver nanoplates (35 nm diameter, lOnm thickness) diluted into 5 mM borate to a concentration of 0.006 mg/mL and 2) 10 nm spherical silver nanoparticles diluted into water to a concentration of 0.006 mg/mL. Borate at 5 mM acts as a stability agent that slows ion release significantly from silver nanoplates providing a negative control for the high ion release rates seen with dilution in water. The 10 nm spherical silver nanoparticles have low curvature and are used for comparison to exemplify the surprising and beneficial effects that arise from silver nanoplates with high curvature. Low curvature 10 nm spherical particles have lower ion release potential despite having higher exposed surface area relative to the silver nanoplates at the same concentration. Low curvature 10 m spherical particles exhibit no color shift during ion release or at expiration. Results are shown in FIG. 11.

### Example 17

### Antimicrobial Gel for Wound Application

A solution of PVP coated silver nanoplates (35 nm diameter, lOnm thickness) in 5 mM borate and water at a concentration of 1.3 mg/mL silver is diluted into a sterile gel consisting of 80% water, 20% propylene glycol, <1% Aristoflex AVC (viscosity 1000 cP) such that the final concentration of silver is 0.13 mg/mL silver. The gel and silver nanoplate solution is spread on the skin over a chronic wound with a 2-4 mm thickness coat. In one example, the wound is dressed with a thin guaze bandage roll (Kendall) and the blue gel soaks through the bandage and forms a visible blue coloring on the dressing. The dressing is maintained moist with daily applications of sterile water. In another example a transparent gel bandage (Second Skin, Spenco) is placed over the wound and antimicrobial gel. After 1 day the gel shifts color to violet and by day 4 the gel in the bandage shifts to red. After 1 week the color of the gel is yellow indicating that the bandage needs to be changed.

### Example 18

### Antimicrobial Adhesive Bandage

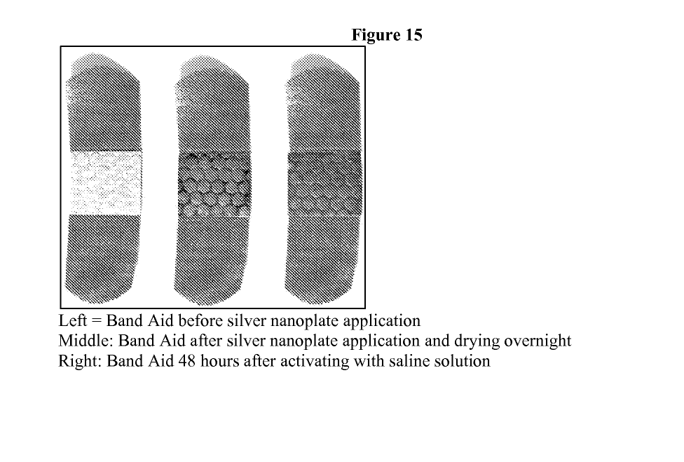

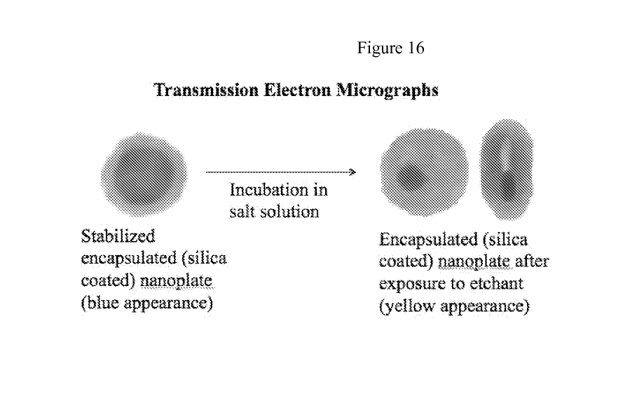

A solution of PVP coated silver nanoplates (35 nm diameter, lOnm thickiness) in 5 mM borate and water at a concentration of 1.3 mg/mL silver is diluted into ethanol to a concentration of 0.13 mg/mL. Approximately 200 microliters of the solution is pipetted onto a 1.5×1.5 cm wound dressing bed on the back of an adhesive strip (Band Aid® adhesive bandage, Johnson & Johnson). The silver nanoplate solution is allowed to dry overnight. Upon drying the nanoplate solution colors the wound dressing bed blue. The adhesive bandage is activated with saline and placed on the skin. Within two hours the wound bed changes to violet signaling activation. After 1 day the wound dressing is red. By 48 hours the wound dressing turns a faint yellow or clear signaling expiration of useful lifetime of the device. Results are shown in FIG. 15.

### Example 19

### Silver Ion Solutions for Laundering

A solution of PVP coated silver nanoplates (35 nm diameter, lOnm thickness) in 5 mM borate water at a concentration of 1.3 mg/mL silver is diluted into a liquid gel carrier containing 5 mM borate water and thickener (Aristoflex AVC) to bring the final concentration of silver in the carrier to 0.013 mg/mL. Approximately 2mL of the carrier with silver nanoplates is diluted into 1 L of water and mixed for 30 seconds with a magnetic stir bar. The diluted carrier is then measured for silver ion content. An aliquot of the dliuted carrier is filtered to separate free ion from nanoplates (CentriPrep filtration device) and the free ion concentration of the diluted carrier is measured by Inductively Coupled Mass Spectrometry to be approximately 10 PPB.

The gelled carrier is added to the wash cycle of a household washing machine and used to wash a white T-shirt (Hanes). After three washes there is no detectable color change in the white T-shirt. Approximately 1 g of the T-shirts is cut away and dissolved in 5 mL of 5% acetic acid. The silver ion content of the T-shirt material is measured by Inductively Coupled Mass Spectrometry to be greater than 5 PPB.

### Example 20

### Silver Ion Tablet for Water Filtration

A solution of PVP coated silver nanoplates (35 nm diameter, lOnm thickness) in 5 mM borate at a concentration of 1.3 mg/mL silver is exchanged into water using a 10 KD dialysis membrane (Pierce, Dialyzer). Additional PVP is added to the silver nanoplates in water at a 1:1 wt/wt ratio with silver and the solution is lyophilized. Flakes containing the PVP and silver nanoplate matrix are scraped from the lyophilized container and 100 ug of the silver nanoplate/PVP flakes are added to 1 L of water. The water is mixed vigorously for 1 min with a magnetic stir bar. Afterwards, free silver ions are filtered from silver nanoplates (CentriPrep filration device) and the silver ion concentration and total silver concentration are measured to be approximately 25 PPM and 50 PPB respectively.

### Example 21

### Silver Foam Dressing with Colorimetric Strikethrough Signaling

A solution of silica coated silver nanoplates in water at a concentration of 2.87 mg/mL silver (˜65 nm in diameter, ˜10 nm in thickness) is synthesized according to Example 2 diluted into water to a concentration of 0.35 mg/mL silver. To a 4×4 cm piece of non-adhesive foam bandage (3 M™ Tegaderm™ Foam Dressing), 1.28 mL of the dilute nanoplate solution is added and manually compressed until the blue-green color on the bandage is homogeneous. The foam bandages are dried at room temperature overnight. The foam bandages are placed in square plastic petri dishes equipped with two small wells filled with water to keep the bandages moist. To the center of a single 4×4 cm foam bandage, different amounts of artificial wound exudate (2% w/v Bovine Serum Albumin, 0.02 M CaCl2, 0.4 M NaCl, 0.08 M tris(hydroxymethyl)aminomethane, pH 7.5) are added. The following amounts of wound exudate are administered via pipette to the center 4 cm2 of the bandage twice daily 6 hours apart for 7 days-124 μL (31 uL/cm2), 248 μL (62 uL/cm2), 500 μL (125 uL/cm2) and 1000 μL (250 uL/cm2). After the first addition of the solutions, the bandages are placed in an incubator set at 37° C. Each day, after the first addition of the etchant solution (wound exudate) a photo is taken of the back of the bandage (see FIG. 17). By Day 7, the foam bandage with the highest loading of etchant has turned entirely yellow, indicating that the population of silver nanoparticles is largely spherical. Strikethrough of the exudate is visualized by color changes in the encapsulated silver nanoplates on various days depending on the rate of exudate. Both initial strikethrough and subsequent color status of the particles are useful to a patient and caregiver in assessing appropriate times to change the bandage.

### Example 22

### Silver Hydrogel Dressing with Colorimetric Signaling

A solution of silica coated nanoplates (35 nm diameter, 10 nm thickness) in water synthesized according to Example 2 is concentrated by centrifugation to a concentration of 16 mg/mL silver. Three grams of isophorone diisocyanate prepolymer was mixed thoroughly first with 5.6 grams of polypropylene glycol (Portion A). Then 8.4 grams of deionized water was mixed with 1.05 grams of propylene glycol, 1.6 grams of polypropylene glycol, and 0.1g of 16 mg/mL silica coated silver nanoplates (Portion B). Portion A and Portion B were mixed thoroughly with a stirring rod for about two to 5 minutes until a homogeneous solution was formed. The solution was then cast into a 10.2 cm×10.2 cm (4″×4″) mold and maintained undisturbed for 90 minutes at room temperature while the gelling reaction occurred. The mold was kept in a closed container at room temperature overnight to prevent water evaporation and to permit essentially complete chemical reaction of all isocyanate end groups. The final hydrogel upon removal from the mold was flexible, transparent and able to absorb water four times, i.e., 400 percent, its own weight. The initial color of the hydrogel was blue. After a 1 day incubation in a saline water bath, the dressing swelled and changed color first to purple, then to red. By 48 hours the dressing had shifted to yellow and by 7 days nearly all of the color was gone.

### Example 23

### Silver Hydrocolloid Dressing with Colorimetric Signaling

The following method was used to make a silver hydrocolloid dressing capable of signaling the activation and progression of silver ion release. Silica encapsulated (Silica coated) silver nanoplates were synthesized according to Example 2, lyophilized, and crushed into a powder. 30-80 parts by weight of medical adhesive polyisobutylene is heated 150° C. and dissolved. Separately, at room temperature, sodium carboxymethyl cellulose (10 parts by weight) pectin (8 parts by weight) and the powder composition of stabilized encapsulated silver nanoplates (0.3 parts by weight) are mixed into a solid powder then stirred slowly into the polyisobutylene mixture with constant stirring under vacuum at a temperature of 140° C., for 60 minutes and then injection molded in a release paper or film I3U. The hydrocolloid is then shaped into a dressing and aged at 35° C. for 80 hours. The initial color of the hydrogel was blue. After a 1 day incubation in a saline water bath, the dressing swelled and changed color from blue to purple, then to red. By 48 hours the dressing had shifted to yellow and by 7 days nearly all of the color was gone.

### Example 24

### IV Administration Set with Continuous Use Indicator

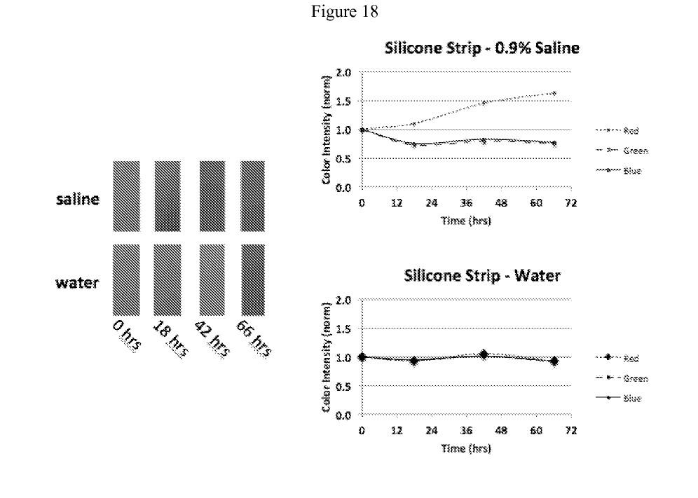

A solution of silica coated nanoplates (35 nm diameter, lOnm thickness) in water synthesized according to Example 2 is concentrated by centrifugation to a concentration of 16 mg/mL silver and mixed with clear or white pigmented silicone prepolymer (Momentum Performance Materials Inc.) at a ratio of 1 part nanoplate solution 40 parts silicone prepolymer. Nanoplate silicone prepolymer is spread on a plastic surface with a microscope slide between two microscope coverslips as space yielding a thin (<1 mm) silicone sheet. The pre-polymer is left to dry at room air overnight. A 2×1 cm strip of nanoplate silicone is cut (see FIG. 18) and sandwiched between two glass plates and additional silicone spacers to form an indicator unit that can be connected to the tubing of a continuous IV drip. Silicone strip has a volume of approximately 50 microliters corresponding to a total silver content of ˜11 micrograms. The continuous indicator unit is inserted in an IV administration set directly beneath the Drip Chamber.

Normal saline from an IV bag is dripped through the IV connector set and continuous use indicator unit for 7 days and the color change of indicator is monitored daily (See FIG. 18). On Day 1, the indicator changes color from blue to purple and by Day 4 the indicator appears red eventually turning orange/yellow by Day 7. CDC guidelines recommend that IV administration sets that are continuously used, including secondary sets and add-on devices, no more frequently than at 96-hour intervals, but at least every 7 days. Thus the indicator provides useful signaling for physicians, nurses, patients, families and other care facilitators on appropriate times to swap out the IV set containing the Continuous Use Indicator.

We estimate less than half of the silver has diffused from the silicone into the IV fluid over 7 days based on the silver nanoplate plasmonic peak wavelength, which corresponds directly with the diameter decrease in the etched silver nanoplates. Even if all of the silver had diffused from the silicone into the IV fluid during 7 days, this would correspond to a daily body absorption of 1.6 micrograms per day. In the United States, the EPA has set a Reference Dose for Chronic Oral Exposure (RfD) of 5 micgrams per Kg per day for silver. RfD is the daily exposure to the human population (including sensitive subgroups) that is likely to be without an appreciable risk of deleterious effects during a lifetime. Oral dose is estimated based on a conversion of 4% absorption of dietary silver into the body. Thus, an intravenous RfD would be approximately 0.2 micrograms per day of silver. For a 70 Kg person, this corresponds to 14 micrograms per day. Thus the continuous use indicator device is delivering nearly an order of magnitude less silver into the body per day than is considered to likely be without an appreciable risk of deleterious effects during a lifetime.

### Example 25

### Silver Impregnated Needless Connector with Colorimetric Signaling

A solution of silica coated nanoplates (35 nm dimeter, 10 nm thickness) in water synthesized according to Example 2 is concentrated by centrifugation to a concentration of 16 mg/mL silver and mixed with clear or white pigmented silicone prepolymer (Momentum Performance Materials Inc.) at a ratio of 1 part nanoplate solution 40 parts silicone prepolymer. Nanoplate silicone prepolymer is spread on a plastic surface with a microscope slide between two microscope coverslips as space yielding a thin (<1 mm) silicone sheet. The silicone connector surface in the lumen of the lure access valve of a needless IV connector set (Maxplus Clear Needless Connector, CareFusion) is pressed onto the thin silicone sheet transferring a thin layer of nanoplate embedded silicone onto the silicone tip in the needless IV connector. The pre-polymer is left to dry at room air overnight. The silica nanoplate embedded silicone strip printed on the surface of the silicone tip of the needless connector has a volume of approximately 10 microliters corresponding to a total silver content of ˜2 micrograms. The needless connector with the nanoplate silicone tip is attached to the male luer of an IV administration set.

Normal saline from an IV bag is flushed through the IV connector set and continuous use indicator unit for 7 days and the color change of indicator is monitored daily by temporarily detaching from the IV set and viewing the silicone tip of the needless connector valve. By Day 1, the indicator changes color from blue to purple and by Day 4 the indicator changes to red eventually turning orange/yellow by Day 7. CDC guidelines recommend that IV administration sets that are continuously used, including secondary sets and add-on devices, no more frequently than at 96-hour intervals, but at least every 7 days. Thus the indicator provides useful signaling for physicians, nurses, patients, families and other care facilitators on appropriate times to swap out the IV connector set. The color is also an important indicator of the status and eventual expiration of antimicrobial activity.

We estimate less than half of the silver has diffused from the silicone into the IV fluid over 7 days based on the silver nanoplate plasmonic peak wavelength, which corresponds directly with the diameter decrease in the etched silver nanoplates. Even if all of the silver had diffused from the silicone into the IV fluid during 7 days, this would correspond to a daily body absorption of 0.3 micrograms per day, significantly less than the the RfD for a 70 Kg person (14 micrograms per day).

### Example 27

### Continuous Use Indicator Unit for Incorporation into IV Sets, Needless Connectors, and Other Components

A solution of silica coated nanoplates (˜30-45 nm diameter, 10 nm thickness) in water synthesized according to Example 2 to have a peak surface plasmon of ˜650 nm and is concentrated by centrifugation to a concentration of 16 mg/mL silver. Particles were loaded a liquid silicone elastomer (NuSil, Self lubricating silicone, MEDI-4955, Lot: 64234) at a loading of 1:20 (16 mg/mL plates:polymer) volumetrically. The particle silicone mixture was then spread between two 15 μm spacers on a plastic sheet to form a thin film. A white backing of silicone mixture with titanium dioxide (no nanoplates) was then applied and the mixture was cured directly on a hot plate for 5 min set to 165° C. A 15 μm top coat of clear silicone mixture (no nanoplates, no titanium dioxide) was then spread over the silicone surface and cured under the same conditions.

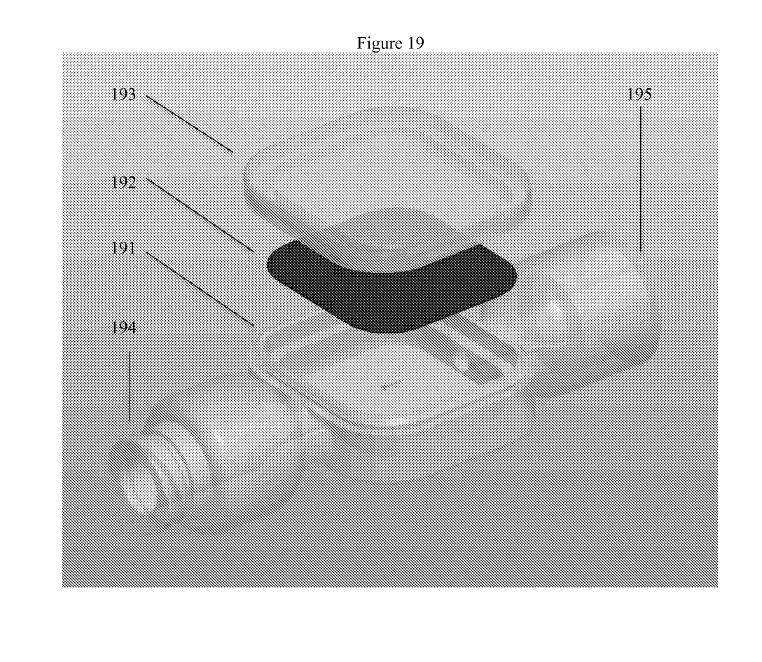

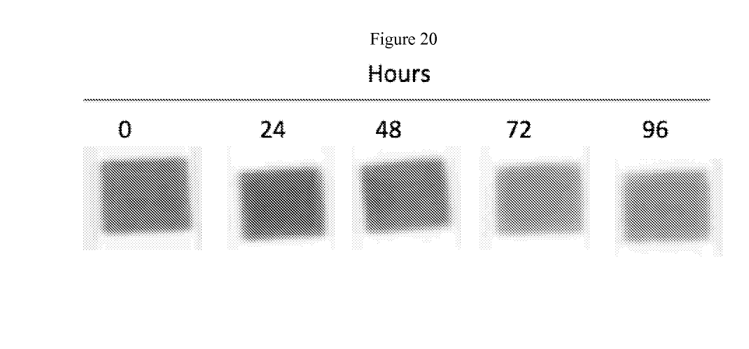

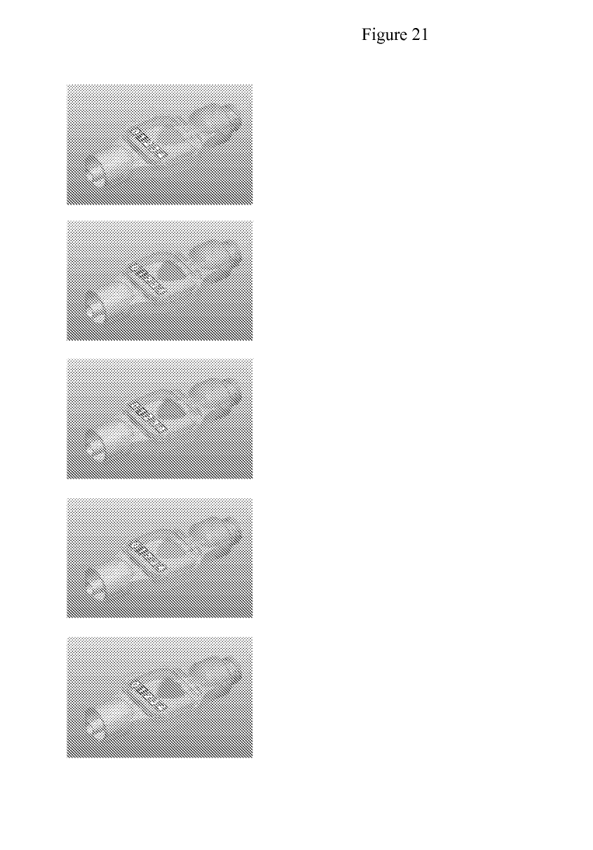

A clear plastic housing was designed using CAD software and printed via a 3D printer (see FIG. 19). The silicone with nanoplate thin strip was cut into a 1×1 cm strip (192) and placed into the continuous use plastic chamber (191 & 193). The plastic chamber was snapped close and connected to the leer lock end of an IV set. Normal saline from an IV bag is dripped through the continuous use indicator unit for 4 days and the color change of the indicator is monitored daily (See FIG. 20). On Day 1, the indicator changes color from blue to purple and by Day 4 the indicator appears red. CDC guidelines recommend that IV administration sets that are continuously used, including secondary sets and add-on devices, no more frequently than at 96-hour intervals, but at least every 7 days. Thus the indicator provides useful signaling for physicians, nurses, patients, families and other care facilitators on appropriate times to swap out the IV set containing the Continuous Use Indicator.

### Example 28

### Body Moisture Indicator for Ulcer Prevention

A solution of silica coated silver nanoplates in water at a concentration of 2.87 mg/mL silver (˜65 nm in diameter, ˜10 nm in thickness) is synthesized according to Example 2 diluted into water to a concentration of 0.35 mg/mL silver. To a 10×10 cm cotton linen sheet, 100 spots of 0.01 mL nanplate solution are pipetted evenly every 1 cm across the linen sheet manually using a pipette man. The linen is dried at room temperature overnight. To various areas of the linen sheet saline is added to mimic body moisture. At various time points over several hours, images are taken of the linen sheet (see FIG. 22). The color changes on the linen over time are useful to a patient or caregiver in detecting moisture to motivate changing an article such as a seat cover or undergarment before pressure ulcers begin to form or worsen.

### Example 29

### Lipoic Acid Coatings to Modulate Silver Ion Release Rates

Silver nanoplates full coated by a monolayer of lipoic acid have high stability in moisture/salt, however it is possible to modulate this stability and speed of ion release rates/particle degradation by only partially coating plates with lipoic acid. PVP coated 580 nm resonant silver nanoplates were synthesized according to Example 1 with subsequent addition of PVP and Borate and concentrated to 1 mg/mL. 0.1 mL of this nanoplate solution was diluted to 0.5 mL with 0.4 mL of 5 mM borate buffer. Lipoic acid solution of 0.01 mg/mL lipoic acid in water was made after reducing with sodium borohydrate. Four mixtures of nanoplates and lipoic acid solutions were made in eppindorf tubes at the following volume ratios A) 0.1 mL nanoplates solution plus 0.01 mL lipoic acid solution, B) 0.1 mL nanoplates solution plus 0.025 mL lipoic acid solution, C) 0.1 mL nanoplates solution plus 0.05 mL lipoic acid solution, and D) 0.1 mL nanoplates solution plus 0.1 mL lipoic acid solution. The solutions were then allowed to incubate for 20 minutes. Then, 5mM borate solution was added to equalize volumes of solutions A-D and the solutions were spun at 10 k rpm for 10 minutes. The supernatant was decanted and pellets were each brought up in 1 mL of water. Solution A immediately started to change from blue to purple in the water solution. Solution B started to change shortly thereafter, but solutions C and D did not change. 0.02 mL of saturated salt was added to solution C and the solution started to change to purple. This experiment demonstrates the ability to vary particle degradations, ion release, and color signaling after exposure to a modulate (e.g., moisture, salt) by varying the monolayer coating density of lipoic acid on silver nanoplates.

1. An article comprising a plurality of plasmonic sifter nanoparticles present at a density effective to provide a colorimetric display,
wherein: a first surface of the article comprises plasmonic silver nanoplates, wherein the silver nanoplates are capable of being contacted with a solvent under conditions such that a plurality of silver ions are released from the silver nanoplates thereby producing a colorimetric display; and
wherein the release of a plurality of silver ions from a plurality of plasmonic silver nanoplates results in a shift of the peak extinction wavelength of the plasmonic silver nanoplates of at least 5 nm.

2-6. (canceled)

7. The article of claim 1, wherein the solvent comprises a biological fluid selected from blood, plasma, serum, wound exudate, sweat, or urine.

8. The article of claim 1, wherein the solvent comprises a salt or an etchant.

9. (canceled)

10. The article of claim 1, wherein the colorimetic display comprises an indicator of: a time of exposure of the article to the solvent, a time of exposure of the article to an environment, a time of exposure of the article to another article, or an expiration of the article.

11-13. (canceled)

14. The article of claim 1, comprising a medical article suitable for application to a human subject.

15. The article of claim 14, wherein the medical article is sterile or is sterilized and wherein the medical article comprises a bandage or dressing.

16. (canceled)

17. The article of claim 15, wherein the silver nanoparticles are disposed on or in a strikethrough detector of the bandage or dressing.

18. The article of claim 1, wherein the silver nanoparticles are dried on a surface.

19. The article of claim 1, wherein the silver nanoparticles are present in a solution, and wherein the silver nanoparticles are substantially stable in the solution.

20. The article of claim 1, wherein the article comprises an intravenous administration set, a needless connector, tubing, an article for pharmaceutical compounding, a moisture detector, a body moisture detector, a food packaging or food preparation article, a food, or a food ingredient.

21-27. (canceled)

28. The article of claim 1 that is capable of indicating the use status of a composition or article of manufacture.

29-44. (canceled)

45. The article of claim 1, wherein:
the silver nanoparticles comprise silver nanoplates that are substantially stable in the absence of moisture,
the silver nanoplates are at a density sufficient to be detectable as a color by an unaided human subject, and
the silver nanoplates are substantially disposed on or in a surface.

46. The article of claim 1, wherein the first surface of the article further comprises a polymer material.

47-49. (canceled)

50. The article of claim 45, wherein the surface comprises a plastic surface, a fiber surface, a glass surface, an absorbable layer, a silicone surface, or an antimicrobial surface.

51-55. (canceled)

56. The article of claim 46, wherein the polymer material comprises a curable polymer, a polyvinyl polymer, or a polystyrene.

57-61. (canceled)

62. The article of claim 45, wherein the silver nanoplates are encapsulated in a metal oxide.

63. The article of claim 62, wherein the metal oxide comprises silica or titanium dioxide and wherein a thickness of the metal oxide is between 2 nm and 100 nm.

64-71. (canceled)

72. The article of claim 1, wherein the silver nanoplates are present on a surface at a surface density of about 0.001 mg to about 1 mg per square inch of the article that is capable of being contacted by a solvent.

73. A method of producing a colorimetric display on an article, comprising the steps of:
i) contacting an article with a solution comprising a plurality of plasmonic silver nanoplates, and
ii) forming a solid under conditions such that the plurality of plasmonic silver nanoplates are disposed on the article at a density effective to provide a colorimetric display, and
wherein:
the silver nanoplates are capable of being contacted with a solvent under conditions such that a plurality of silver ions are released from the silver nanoplates, and
the colorimetric display comprises a visible spectrum color change upon release of a pluralit of silver ions from the silver nanoplates.

74-77. (canceled)

78. The method of claim 73, wherein the solid is formed by evaporation of a solvent from the solution.

79. The method of claim 73, wherein the solution comprises a curable liquid.
